(19) United States
(12) Patent Application Publication
(10) Pub. No.: US 2025/0034457 A1
(43) Pub. Date: Jan. 30, 2025

(54) WASTE PLASTIC-BASED THERMAL CRACKING FEED AND METHOD FOR UPGRADING THE SAME

(71) Applicant: Neste Oyj, Espoo (FI)

(72) Inventors: John JAMIESON, Porvoo (FI); Antti OJALA, Porvoo (FI); Valeria MORELIUS, Porvoo (FI); Ville PAASIKALLIO, Porvoo (FI)

(73) Assignee: Neste Oyj, Espoo (FI)

(21) Appl. No.: 18/715,779

(22) PCT Filed: Sep. 7, 2022
(86) PCT No.: PCT/EP2022/074849

§ 371 Date: Jun. 3, 2024

(30) Foreign Application Priority Data
Dec. 3, 2021 (FI) 20216242

(51) Int. Cl. C10G 9/36; C07C 4/04

(52) U.S. Cl. CPC C10G 9/36; C07C 4/04; C10G 2300/1003; C10G 2300/1092

(57) ABSTRACT

The disclosure relates to a method for upgrading liquefied waste plastic, wherein the method includes a step of providing a liquefied waste plastic feed, a step of thermally cracking the liquefied waste plastic feed in a thermal cracking reactor, and a step of separating at least a fraction including ethylene from the effluent of the thermal cracking reactor, wherein the liquefied waste plastic feed has a content ratio of i-olefins to n-olefins of 3.0 or less. The disclosure also provides a liquefied waste plastic feed for thermal cracking.

### TECHNICAL FIELD

The present invention generally relates to a method for upgrading liquefied waste plastic, and specifically relates to a thermal cracking feed based on liquefied waste plastic and to a method of thermally cracking this feedstock and separating at least a fraction comprising ethylene from the cracking effluent, and further to products obtained in this procedure.

### BACKGROUND OF THE INVENTION

The purification of liquefied waste plastics (LWP) to yield more valuable (or pure) substances and the conversion of liquefied waste plastics into more valuable material, such as light olefins which can be used as raw material in the chemical industry, have been studied for several years.

LWP is a product of depolymerisation of waste plastic and is typically produced by hydrothermal liquefaction (HTL) or pyrolysis of waste plastics. Depending on the source of the waste plastics, LWP has variable levels of impurities. Typical impurity components include chlorine, nitrogen, sulphur and oxygen. These impurities are also common in post-consumer packaging waste which is a large scale source for plastics waste.

Besides use as a fuel, chemical recycling of LWP back to plastic or to monomers is an interesting option which has sparked significant interest in the petrochemical industry during the last years. Also for this route, some of the impurities in LWP may be detrimental and they have to be at least partly removed.

In addition to the impurity aspects there are differences in compositional features of the LWP such as olefins, aromatics and the presence of structural isomers that can also affect the performance of the monomers, e.g. ethylene, propylene or butylene, production process such as variations in primary product yields or enhancing the coking tendency leading to reduced run lengths.

For example, depending on the actual production method, LWP has high to very high total olefins content. Such olefins are usually considered as coke-promotors in thermal conversion processes, such as steam cracking, and are conventionally removed, e.g. by total hydrogenation. This procedure, however, consumes a high amount of hydrogen. Since hydrogen production is a significant CO₂ emitter and an energy intensive production unit at refinery, avoiding hydrogen consumption both avoids cost and is more sustainable.

In view of the increasing interest in recycling of plastic waste, several routes of upgrading LWP have been developed.

U.S. Pat. No. 5,731,483 A discloses a process for recycling plastic waste in a steam cracker, wherein a melted plastic waste is heated to 400-550° C. and a distillate fraction is fed to a steam cracker.

U.S. Pat. No. 5,639,937 A discloses a process for the production of light olefins from plastic waste, comprising thermal pre-processing of waste plastic to adjust viscosity thereof and subsequent thermal treatment at 700-1100° C.

WO 2021/110395 A1 discloses a two-step hydrotreatment method to minimize impurities, such as olefins, in a liquefied waste plastic feed before steam cracking.

WO 2018/025104 A1 discloses a process for processing mixed waste plastic comprising simultaneous pyrolysis and dechlorination of the mixed waste plastics to provide a hydrocarbon gas stream and a hydrocarbon liquid stream. The hydrocarbon liquid stream may be hydroprocessed to minimize the olefins content thereof. The hydroprocessed hydrocarbon liquid stream may be subjected to steam cracking.

EP 3110912 B1 discloses a process for converting waste plastic into petrochemicals, comprising pyrolysis of waste plastic to obtain a liquid stream, separating said liquid stream into a stream having high aromatics content and a stream having low aromatics content and sending the stream having low aromatics content to a hydrocracking unit.

### BRIEF DESCRIPTION OF THE DRAWING

FIG. 1 is a schematic diagram illustrating a bench scale thermal cracking apparatus as employed in the Examples.

### Brief description of the invention

The present invention was made in view of the above-mentioned problems and it is an object of the present invention to provide an improved method for upgrading liquefied waste plastic as well as a thermal cracking feed based on waste plastic for use in such a method.

This problem of providing an improved method for upgrading liquefied waste plastic is solved by a method of claim 1. Further beneficial developments are set forth in subsequent claims.

In brief, the present invention relates to one or more of the following items:
1. A method for upgrading liquefied waste plastic, wherein the method comprises
(a) a step of providing a liquefied waste plastic feed,
(b) a step of thermally cracking the liquefied waste plastic feed in a thermal cracking reactor, and
(c) a step of separating at least a fraction comprising ethylene from the effluent of the thermal cracking reactor,
   characterized in that
   the liquefied waste plastic feed has a content ratio of i-olefins to n-olefins of 3.0 or less.
2. The method according to item 1, wherein the liquefied waste plastic feed has a content ratio of i-olefins to n-olefins of 2.5 or less, 2.0 or less, 1.5 or less, 1.0 or less, 0.80 or less, 0.60 or less, or 0.50 or less.
3. The method according to any one of the preceding items, wherein the liquefied waste plastic feed has a total content of i-olefins, n-olefins, i-paraffins and n-paraffins of at least 40 wt.-%, preferably at least 50 wt.-%, at least 55 wt.-%, at least 60 wt.-%, at least 65 wt.-% or at least 70 wt.-%.
4. The method according to any one of the preceding items, wherein the liquefied waste plastic feed is an optionally pre-treated fraction of liquefied waste plastic or a combination of two or more optionally pre-treated fractions of liquefied waste plastic.
5. The method according to any one of the preceding items, wherein the liquefied waste plastic feed is an optionally pre-treated degassed fraction of liquefied waste plastic.
6. The method according to any one of the preceding items, wherein the liquefied waste plastic feed is an optionally pre-treated liquid or waxy fraction of liquefied waste plastic or a combination thereof.
7. The method according to any one of the preceding items, wherein the liquefied waste plastic feed is an optionally pre-treated naphtha fraction of liquefied waste plastic or an optionally pre-treated middle distillate fraction of liquefied waste plastic, preferably an optionally pre-treated naphtha fraction of liquefied waste plastic.
8. The method according to any one of the preceding items, wherein the liquefied waste plastic feed comprises non-gaseous (NTP) hydrocarbons of liquefied waste plastic.
9. The method according to any one of the preceding items, wherein the liquefied waste plastic feed has a total content of gaseous (NTP) components of 1.5 wt.-% or less, preferably 1.0 wt.-% or less, more preferably 0.5 wt.-% or less.
10. The method according to any one of the preceding items, wherein the liquefied waste plastic feed has a content of solid components of 1000 wt.-ppm or less, preferably 100 wt.-ppm or less, more preferably 10 wt.-ppm or less.
11. The method according to any one of the preceding items, wherein the liquefied waste plastic feed has a total olefins content of 10 wt.-% or more, such as 15 wt.-% or more, 20 wt.-% or more or 30 wt.-% or more.
12. The method according to any one of the preceding items, wherein the liquefied waste plastic feed has a total olefins content of 90 wt.-% or less, preferably 80 wt.-% or less, 70 wt.-% or less or 60 wt.-% or less.
13. The method according to any one of the preceding items, wherein the liquefied waste plastic feed has a content ratio of monoolefins to diolefins of 10 or more, preferably 12 or more, 15 or more or 20 or more.
14. The method according to any one of the preceding items, wherein the liquefied waste plastic feed has a content ratio of i-paraffins to n-paraffins of 2.0 or less, preferably 1.7 or less, 1.5 or less, 1.0 or less, or 0.90 or less.
15. The method according to any one of the preceding items, wherein the liquefied waste plastic feed has a content ratio of i-paraffins to n-paraffins of 0.80 or less, such as 0.70 or less, 0.60 or less, 0.50 or less, 0.40 or less, 0.30 or less, 0.20 or less, 0.15 or less, 0.10 or less, 0.09 or less, 0.08 or less, 0.07 or less, 0.06 or less, 0.05 or less, 0.04 or less.
16. The method according to any one of the preceding items, wherein the liquefied waste plastic feed comprises optionally pre-treated liquefied sorted waste plastic or a fraction thereof.
17. The method according to any one of the preceding items, wherein the liquefied waste plastic feed comprises optionally pre-treated liquefied polyolefin-rich waste plastic or a fraction thereof.
18. The method according to any one of the preceding items, wherein the liquefied waste plastic feed comprises optionally pre-treated liquefied polyethylene-rich waste plastic or a fraction thereof.
19. The method according to any one of the preceding items, wherein the liquefied waste plastic feed has a total paraffins content of 5 wt.-% or more, preferably 10 wt.-% or more, 12 wt.-% or more, 15 wt.-% or more, 17 wt.-% or more, 19 wt.-% or more, or 20 wt.-% or more.
20. The method according to any one of the preceding items, wherein the liquefied waste plastic feed has a total monoaromatics content of 30 wt.-% or less, preferably 25 wt.-% or less, 20 wt.-% or less, or 15 wt.-% or less
21. The method according to any one of the preceding items, wherein the liquefied waste plastic feed has a total content of n-paraffins and n-olefins of 20 wt.-% or more, preferably 23 wt.-% or more, 25 wt.-% or more, 27 wt.-% or more, or 30 wt.-% or more.
22. The method according to any one of the preceding items, wherein the liquefied waste plastic feed has a content ratio of total n-paraffins and n-olefins to total i-paraffins and i-olefins ([nP]+[nO])/([iP]+[iO]) of 0.40 or more, preferably 0.50 or more, 0.60 or more, 0.70 or more, 0.80 or more, 0.90 or more, 1.00 or more, 1.20 or more, 1.40 or more, 1.60 or more, 1.80 or more, 2.00 or more, 2.50 or more, or 3.00 or more.
23. The method according to any one of the preceding items, wherein the liquefied waste plastic feed has a total content of n-olefins, i-olefins and diolefins ([nO]+[iO]+[diO]) of 60 wt.-% or less, preferably 55 wt.-% or less, or 50 wt.-% or less.
24. The method according to any one of the preceding items, wherein the liquefied waste plastic feed has a content ratio of total n-olefins and i-olefins to diolefins (([nO]+[iO])/[diO]) of 5 or more, preferably 8 or more, or 10 or more.
25. The method according to any one of the preceding items, wherein the following equation is fulfilled:

$$[ iO ] * [ diO ] < α$$

wherein [iO] is the content of i-olefins in the liquefied waste plastic feed and [diO] is the content of diolefins in the liquefied waste plastic feed, and a is 100, preferably 90, 80, or 75.
26. The method according to any one of the preceding items, wherein the following equation is fulfilled:

$$( [ iO ] + 2 * [ iP ] ) * [ diO ] < β$$

wherein [iO] is the content of i-olefins in the liquefied waste plastic feed, [diO] is the content of diolefins in the liquefied waste plastic feed, [iP] is the content of i-paraffins in the liquefied waste plastic feed, and β is 150, preferably 130, 120, 100, 90, 80, or 75.
27. The method according to any one of the preceding items, wherein the liquefied waste plastic feed has a total content of i-paraffins and i-olefins of 50 wt.-% or less, preferably 40 wt.-% or less, 30 wt.-% or less, or 20 wt.-% or less.
28. The method according to any one of the preceding items, wherein the liquefied waste plastic feed has a T5 temperature (5 vol-% recovered, EN ISO 3405-2019) of 20° C. or more, preferably 25° C. or more, or 30° C. or more.
29. The method according to any one of the preceding items, wherein the liquefied waste plastic feed has a total content of C9 i-olefins of 10 wt.-% or less, preferably 8 wt.-% or less, 6 wt.-% or less or 5 wt.-% or less.
30. The method according to any one of the preceding items, wherein a fraction of the liquefied waste plastic feed having a T95 temperature (95 vol-% recovered, EN ISO 3405-2019) of 180° C. or less, preferably 165° C. or less, has a total content of C9 i-olefins of 10 wt.-% or less, preferably 8 wt.-% or less, 6 wt.-% or less or 5 wt.-% or less
31. The method according to any one of the preceding items, wherein the liquefied waste plastic feed has a content of diolefins of 5.0 wt.-% or less, preferably 4.0 wt.-% or less, 3.0 wt.-% or less, 2.5 wt.-% or less, 2.0 wt.-% or less, 1.5 wt.-% or less or 1.0 wt.-% or less.
32. The method according to any one of the preceding items, wherein the liquefied waste plastic feed has a content of conjugated diolefins of 5.0 wt.-% or less, more preferably 4.0 wt.-% or less, 3.0 wt.-% or less, 2.0 wt.-% or less, 1.0 wt.-% or less, 0.75 wt.-% or less or 0.50 wt.-% or less.
33. The method according to any one of the preceding items, wherein the liquefied waste plastic feed has an acidity of 10.0 mgKOH/g or less, preferably 8.0 mgKOH/g or less, 6.0 mgKOH/g or less, 4.0 mgKOH/g or less, 2.0mgKOH/g or less, 1.5 mgKOH/g or less, 1.0 mgKOH/g or less, 0.9 mgKOH/g or less, 0.8 mgKOH/g or less, 0.7 mgKOH/g or less, 0.6 mgKOH/g or less, or 0.5 mgKOH/g or less.
34. The method according to any one of the preceding items, wherein the liquefied waste plastic feed has an oxygen content of 1.50 wt.-% or less, preferably 1.40 wt.-% or less, 1.30 wt.-% or less, 1.20 wt.-% or less, 1.10wt.-% or less, 1.00 wt.-% or less, 0.90 wt.-% or less, or 0.80 wt.-% or less.
35. The method according to any one of the preceding items, wherein the liquefied waste plastic feed is obtained by depolymerisation of waste plastic, followed by gas-liquid separation and optional further fractionation and optional further pre-treatment.
36. The method according to any one of the preceding items, wherein the liquefied waste plastic feed comprises a waste plastics pyrolysis oil (WPPO) or a fraction thereof.
37. The method according to any one of the preceding items, wherein the liquefied waste plastic feed comprises a waste plastic hydrothermal liquefaction (HTL) oil, or a fraction thereof.
38. The method according to any one of the preceding items, wherein thermal cracking in step (b) comprises steam cracking.
39. The method according to any one of the preceding items, wherein the feed to the thermal cracking reactor comprises sulphur 20-300 ppm by weight, preferably 20-250 ppm by weight, more preferably 20-100 ppm by weight, and even more preferably 50-65 ppm by weight.
40. The method according to any one of the preceding items, wherein the thermal cracking step (b) is conducted at a coil outlet temperature (COT) selected from the range from 750° C. to 920° C., preferably from 750° C. to 890° C., more preferably from 800° C. to 880° C., and even more preferably from 820° C. to 860° C.
41. The method according to any one of the preceding items, wherein the thermal cracking step (b) is conducted at a coil outlet pressures (COP) within a range from 1.3 bar to 6.0 bar, preferably from 1.3 bar to 3.0 bar.
42. The method according to any one of the preceding items, wherein the thermal cracking step (b) is conducted at a flow rate ratio between cracking diluent, preferably steam, and the total cracker feed, expressed as flow rate of diluent [kg/h]/flow rate of total cracker feed [kg/h], within a range from 0.10 to 1.00, preferably from 0.25 to 0.85.
43. The method according to any one of the preceding items, wherein the step (c) comprises recovering a recycle stream from the thermal cracking effluent and, after one or more optional hydrotreatment(s) and/or further post-processing(s), subjecting the recycle stream to thermal cracking in the thermal cracking step (b) as a co-feed with the LWP feed.
44. The method according to any one of the preceding items, wherein the step (c) further comprises separating at least a fraction comprising propylene from the effluent of the thermal cracking reactor.
45. The method according to any one of the preceding items, comprising within step (c) a stage of subjecting at least a portion of the thermal cracking effluent to a purification treatment to remove at least one of CO, CO₂, MAPD, and C₂H₂.
46. The method according to any one of the preceding items, comprising
   providing multiple thermal cracker furnaces, and
   performing the thermal cracking step (b) in at least one of the multiple thermal cracker furnaces.
47. The method according to the preceding item, comprising
   obtaining cracking effluents from the multiple thermal cracking furnaces, optionally post-treating at least one of the thermal cracking effluents, and
   mixing the obtained thermal cracking effluents to form a combined cracking product, and
   optionally subjecting at least a portion of the combined cracking product to a purification treatment to remove at least one of CO, CO₂, MAPD and C₂H₂.
48. The method according to any one of the preceding items, wherein the thermal cracking in step (b) is carried out in the presence of a co-feed.
49. The method according to any one of the preceding items, wherein the thermal cracking in step (b) is carried out in the presence of a hydrocarbon co-feed.
50. The method according to any one of the preceding items, wherein the thermal cracking in step (b) is carried out in the presence of a fossil hydrocarbon co-feed, preferably a fossil naphtha co-feed, more preferably a fossil naphtha feed selected from full range naphtha or light naphtha.
51. The method according to any one of the two preceding items, wherein the co-feed is a fossil naphtha feed fulfilling the following standard specifications:
   Specific gravity at 15° C. is at most 0.735 g/ml,
   Reid Vapour Pressure is at most 12.5 psi,
   Colour is at least +20 Saybolt,
   Initial Boiling point is at least 30° C.,
   Final Boiling point is at most 180° C.,
   Paraffins content is at least 65% by volume, Olefins content is at most 1% by volume, the balance being naphthenes and aromates,
   Sulfur content is at most 500 ppm,
   H2S content is at most 10 ppm,
   Lead content is at most 50 ppb,
   content of organic chlorides is at most 3 ppm,
   MTBE content is at most 50 ppm,
   Total oxygenates content is at most 100 ppm,
   Mercury content is at most 5 ppb.
52. The method according to any one of the preceding items, wherein the thermal cracking in step (b) is carried out in the presence of a co-feed having a total content of aromatics and naphthenes of at most 35 wt.-%, preferably at most 30 wt.-%, at most 25 wt.-%, at most 20 wt.-%, at most 15 wt.-% or at most 10 wt. %.
53. The method according to any one of the preceding items, the thermal cracking in step (b) is carried out in the presence of a co-feed having a total olefins content of at most 3.0 wt.-%, preferably at most 1.0 wt.-%, at most 0.75 wt.-%, at most 0.50 wt.-%, at most 0.30 wt.-% or at most 0.10 wt. %.
54. The method according to any one of the preceding items, wherein the thermal cracking in step (b) is carried out in the presence of a co-feed having a total oxygenates content of at most 1.0 wt.-%, preferably at most 0.50 wt.-%, at most 0.30 wt.-%, at most 0.10 wt. %, or at most 0.05 wt.-%.
55. The method according to any one of the preceding items, wherein the thermal cracking in step (b) is carried out in the presence of a renewable hydrocarbon co-feed, such as a hydrotreated vegetable oil and/or animal fat (HVO) hydrocarbon co-feed.
56. The method according to any one of the preceding items, wherein the thermal cracking in step (b) is carried out in the presence of a Fischer-Tropsch hydrocarbon co-feed.
57. The method according to any one of the preceding items, wherein the step of providing the liquefied waste plastic feed comprises a stage of depolymerizing solid waste plastic to provide liquefied waste plastic.
58. The method according to item 57, wherein the step of providing the liquefied waste plastic feed further comprises a stage of pre-processing solid waste plastic.
59. The method according to item 58, wherein the pre-processing of solid waste plastic comprises separating the solid waste plastic from other waste, such as paper, metal and/or compostable waste.
60. The method according to any one of items 58 to 59, wherein the pre-processing of solid waste plastic comprises drying the solid waste plastic.
61. The method according to any one of items 58 to 60, wherein the pre-processing of solid waste plastic comprises reducing the size of the solid waste plastic, such as cutting the solid waste plastic, shredding the solid waste plastic and/or comminuting the solid waste plastic.
62. The method according to any one of items 58 to 61, wherein the pre-processing of solid waste plastic comprises melting the solid waste plastic.
63. The method according to any one of items 57 to 62, wherein the stage of depolymerizing solid waste plastic comprises thermal depolymerisation to provide waste plastic thermal depolymerisation oil.
64. The method according to any one of items 57 to 63, wherein the stage of depolymerizing solid waste plastic comprises hydrothermal liquefaction to provide waste plastic hydrothermal liquefaction oil.
65. The method according to any one of items 57 to 64, wherein the stage of depolymerizing solid waste plastic comprises thermal pyrolysis to provide waste plastic pyrolysis oil (WPPO), wherein the thermal pyrolysis may be thermal non-catalytic pyrolysis or thermal catalytic pyrolysis.
66. The method according to any one of items 57 to 65, wherein the liquefied waste plastic feed comprises depolymerized solid waste plastic, preferably at least one of waste plastic pyrolysis oil (WPPO) or hydrothermal liquefaction oil from solid waste plastic.
67. The method according to any one of items 57 to 66, wherein the solid waste plastic comprises sorted waste plastic.
68. The method according to any one of items 57 to 67, wherein the solid waste plastic comprises polyolefin-rich waste plastic.
69. The method according to any one of items 57 to 68, wherein the solid waste plastic comprises polyethylene-rich waste plastic.
70. The method according to any one of items 57 to 69, wherein the solid waste plastic has an oxygen content of 15 wt.-% or less, preferably 10 wt.-% or less, more preferably 5 wt.-% or less, determined as elemental O, of the total weight of the solid waste plastic.
71. The method according to any one of items 57 to 70, wherein the solid waste plastic has ratio between the melting enthalpy in the temperature range of 70-130° C. and the melting enthalpy in the temperature range of 130-180° C., E_{70-130° C.}/E_{130-180° .}, as measured by DSC, is 50% or more, preferably 60% or more or 70% or more.
72. The method according to any one of items 57 to 71, wherein the solid waste plastic has an intensity ratio between the intensity I₁₁₆₈ of the FT-IR peak at 1168 cm⁻¹, and the total intensities I₁₁₆₈+I₇₁₆ of peaks at 1168 cm⁻¹and 716 cm⁻¹, I_{1168/[}I₁₁₆₈+I₇₁₆], is 0.3 or less, preferably 0.2 or less.
73. The method according to any one of the preceding items, wherein the step of providing the liquefied waste plastic feed comprises a stage of separating at least gaseous (NTP) components from liquefied waste plastic.
74. The method according to any one of the preceding items, wherein the step of providing the liquefied waste plastic feed comprises a stage of separating at least gaseous (NTP) components from liquefied waste plastic and recovering at least one gaseous (NTP) olefin species, preferably at least ethylene, from the gaseous (NTP) components.
75. The method according to the preceding item, further comprising combining ethylene recovered from the gaseous (NTP) components with the fraction obtained in step (c) or with a purified ethylene fraction derived from the fraction obtained in step (c).
76. The method according to any one of the preceding items, wherein the step (a) of providing the liquefied waste plastic feed comprises a stage of fractionating liquefied waste plastic, preferably yielding a middle distillate fraction and/or a naphtha fraction, more preferably a naphtha fraction as the liquefied waste plastic feed.
77. The method according to any one of the preceding items, wherein the step (a) of providing the liquefied waste plastic feed comprises a stage of separating high-boiling components from liquefied waste plastic, preferably separating components having a boiling point of more than 450° C.
78. The method according to any one of the preceding items, wherein the step (a) of providing the liquefied waste plastic feed comprises at least one pre-treatment stage in which liquefied waste plastic or a fraction thereof is subjected to pre-treatment.
79. The method according to item 78, wherein the pre-treatment comprises drying the liquefied waste plastic, such as drying by means of phase separation, evaporation, and/or moisture absorbents.
80. The method according to any one of items 78 to 79, wherein the pre-treatment comprises subjecting the liquefied waste plastic to at least one treatment selected from the group consisting of desalting, reactive extraction, solvent extraction, adsorption, oxidation, and reduction.
81. The method according to any one of items 78 to 80, wherein the pre-treatment comprises separation of solid particles, such as char, from the liquefied waste plastic, such as separation by means of settling, decanting, centrifugation and/or filtering.
82. The method according to any one of items 78 to 81, wherein the pre-treatment comprises separation of gaseous (NTP) components from the liquefied waste plastic, such gas-liquid separation and/or fractionation.
83. The method according to any one of items 78 to 82, wherein the pre-treatment comprises selective and/or mild hydrogenation of the liquefied waste plastic, such selective and/or mild hydrogenation of dienes and/or alkynes to monoolefins or alkanes and/or selective and/or mild hydrogenation of MAPD to monoolefins or alkanes.
84. The method according to any one of the preceding items, wherein the liquefied waste plastic feed has a content of n-olefins of 2 wt.-% or more, preferably 5 wt.-% or more, 10 wt.-% or more, 15 wt.-% or more, 20 wt.-% or more, or 25 wt.-% or more.
85. The method according to any one of the preceding items, wherein the liquefied waste plastic feed has a total aromatics content of 35 wt.-% or less, preferably 30 wt.-% or less, 25 wt.-% or less, 20 wt.-% or less, or 15 wt.-% or less.
86. A liquefied waste plastic feed for thermal cracking, wherein the liquefied waste plastic feed has a content ratio of i-olefins to n-olefins of 3.0 or less.
87. The liquefied waste plastic feed for thermal cracking according to item 86, wherein the liquefied waste plastic feed comprises an optionally pre-treated liquefied waste plastic or a fraction thereof, such as an optionally pre-treated waste plastic thermally depolymerized oil or a fraction thereof.
88. A liquefied waste plastic feed for thermal cracking, wherein the liquefied waste plastic feed is an optionally pre-treated liquefied waste plastic obtained from a process comprising depolymerisation of polyethylene-rich waste plastic, or a fraction thereof.
89. The liquefied waste plastic feed according to any one of items 86 to 88, further having characteristic(s) as defined for the liquefied waste plastic feed in any one of items 1 to 85.

### DETAILED DESCRIPTION OF THE INVENTION

The present invention relates to a method for upgrading liquefied waste plastic. The method comprises a step of providing a liquefied waste plastic feed (step a), a step of thermally cracking the liquefied waste plastic feed in a thermal cracking reactor (step b), and a step of separating at least a fraction comprising ethylene from the effluent of the thermal cracking reactor (step c). In accordance with the invention, the liquefied waste plastic feed has a content ratio of i-olefins to n-olefins of 3.0 or less.

In the present invention, liquefied waste plastic (LWP) means a product effluent from liquefaction process obtainable by depolymerizing waste plastics. LWP usually has a high total olefins content. Olefins are conventionally considered as a main cause for coking in down-stream processing. Conventionally, LWP was subjected to severe hydrogenation of olefins to paraffins, such as total hydrogenation, or other workup before being processed further, in order to reduce coking tendency.

The present inventors now surprisingly found that i-olefins content correlates with yields of ethylene and propylene and that high yield of, in particular, ethylene can be achieved when thermally cracking a liquefied waste plastic feed having low i-olefins content. The present inventors furthermore found that conjugated diolefins are particularly strong coke promoters.

Based on this finding, the present invention provides an improved method which requires no hydrogenation or less hydrogenation but nevertheless achieves high yields of valuable products, in particular ethylene.

The liquefied waste plastic feed of the present invention is derived from liquefied waste plastic. Liquefied waste plastic means a product effluent from a liquefaction process comprising at least depolymerising waste plastics. The liquefaction process is typically carried out at elevated temperature, and preferably under non-oxidative conditions. The liquefaction process may be carried out at elevated pressure. The liquefaction process may be carried out in the presence of a catalyst. The effluent from the liquefaction process or a fraction thereof may be employed as the liquefied waste plastic feed as such or may preferably be subjected to at least one pre-treatment to provide the liquefied waste plastic feed. Suitable pre-treatments are described below. In the present invention, when reference is made to the material constituting the liquefied waste plastic feed, this is meant to encompass the pre-treated material as well. In other words, a material constituting the liquefied waste plastic feed shall mean an optionally pre-treated material constituting the liquefied waste plastic feed. For example, when stating that the LWP feed is a hydrothermal liquefaction oil or a fraction thereof, this encompasses pre-treated hydrothermal liquefaction oil, a pre-treated fraction of hydrothermal liquefaction oil, a fraction of pre-treated hydrothermal liquefaction oil and a pre-treated fraction of pre-treated hydrothermal liquefaction oil. In other words, the optional pre-treatment may be accomplished before fractionation or after fractionation or both. Similarly, multiple fractionations may be carried out. In addition, two or more optionally pre-treated fractions of LWP may be combined to give the LWP feed. The two or more fractions may have the same or similar boiling range or may have different boiling ranges.

In addition to liquid (NTP) hydrocarbons, i.e. hydrocarbons being liquid at normal temperature and pressure (NTP; 20° C., 101.325 kPa absolute), typical product effluents from liquefaction processes comprise gaseous (NTP) hydrocarbons, and hydrocarbons that are waxy or solid at NTP but become liquids upon heating, for example upon heating to 80° C.

In the context of the present disclosure, depolymerizing waste plastic means decomposing or degrading the polymer backbones of the waste plastic, typically at least thermally, to the extent yielding polymer and/or oligomer species of smaller molecular weight compared to the starting waste plastic, but still comprising at least liquid (NTP) hydrocarbons. In other words, as used herein, the liquefied waste plastic does not cover plastics in liquid form obtained merely by melting or by dissolving into a solvent, as these do not involve sufficient cleavage of the polymer backbones, nor waste plastics depolymerized completely to the monomer-level and thus being of gaseous (NTP) form. Depolymerizing waste plastics may also involve cleavage of covalently bound heteroatoms such as O, S, and N from optionally present heteroatom-containing compounds.

Initially the waste plastic, or each waste plastic species in mixed waste plastics, to be subjected to liquefaction, is in solid state, typically having a melting point in the range of 100° C. or more as measured by DSC as described by Larsen et al in the citation below. However, the waste plastic, or each waste plastic species, may be melted before and/or during the depolymerisation.

Solid waste plastics may contain various further components such as additives, reinforcing materials, etc., including fillers, pigments, printing, inks, flame retardants, stabilizers, antioxidants, plasticizers, lubricants, labels, metals, paper, board, cellulosic fibres, fibre-glass, even sand or other dirt. Some of the further components may be removed, if so desired, from the solid waste plastics, from melted waste plastic, and/or from liquefied waste plastic using commonly known methods.

Preferably, the solid waste plastics to be subjected to the depolymerisation, and thus being the base material of the LWP feed, has an oxygen content of 15 wt.-% or less, preferably 10 wt.-% or less, more preferably 5 wt.-% or less, of the total weight of the solid waste plastics. The oxygen content may be 0 wt.-% and may preferably be in the range of 0 wt.-% to 15 wt.-% or 0 wt.-% to 10 wt. %. Oxygen content in wt.-% can be determined by difference using the formula 100 wt.-%-(CHN content+ash content), wherein CHN content refers to combined content of carbon, hydrogen and nitrogen, as determined in accordance with ASTM D5291, and ash content refers to ash content as determined in accordance with ASTM D482/EN15403. In the present disclosure, all standards refer to the latest version available on Dec. 1, 2021, unless stated to the contrary.

The liquefied waste plastic obtainable by depolymerizing waste plastic comprises primarily hydrocarbons, typically more than 50 wt.-% based on the total weight of the LWP. Typically the LWP comprises two or more hydrocarbon species selected from paraffins, olefins, naphthenes and aromatics. The composition of the LWP may vary depending e.g. on the composition of the solid waste plastic, liquefaction process type and conditions, and any additional treatments. Further, the assortment of various species of solid waste plastics and impurities associated with collected waste may result in a presence of impurities including silicon, sulphur, nitrogen, halogens and oxygen related substances in various quantities in the LWP.

The liquefied waste plastic feed of the present invention is not particularly limited as long as it is has a content ratio of i-olefins to n-olefins of 3.0 or less.

In the present invention, unless specified otherwise, a “content ratio” refers to the ratio on a weight basis of the contents of the two components. That is, in accordance with claim 1, the liquefied waste plastic feed having a content ratio of i-olefins to n-olefins of 3.0 or less means that the content by weight of i-olefins in the liquefied waste plastic feed divided by the content by weight of n-olefins in the liquefied waste plastic feed is 3.0 or less.

In the present invention, unless otherwise specified, a “content” is a content on a weight basis and is calculated relative to the total weight of the base material, such as the total weight of the liquefied waste plastic feed.

In the present invention, i-olefins refer to branched non-cyclic monoolefins and n-olefins refer to linear non-cyclic monoolefins. In the present specification, i-olefins may also be referred to as iso-olefins and n-olefins may also be referred to as normal-olefins. All non-aromatic compounds having at least one carbon-carbon double bond are collectively referred to as “total olefins” or simply “olefins”. Thus, the total olefins content refers to the summed content of all compounds having at least one carbon-carbon double bond.

The liquefied waste plastic feed preferably has a content ratio of i-olefins to n-olefins of 2.5 or less. The content ratio is more preferably 2.0 or less, 1.5 or less, 1.0 or less, 0.80 or less, 0.60 or less, or 0.50 or less. Specifically, the liquefied waste plastic feed may have a content ratio of i-olefins to n-olefins of from 0 to 3.0, preferably 0 to 2.0, 0 to 1,5, 0.01 to 1.0, 0.02 to 0.80, 0.03 to 0.60 or 0.05 to 0.50.

A low content ratio of i-olefins to n-olefins further improves the ethylene yield and may reduce coking tendency.

The liquefied waste plastic feed preferably has a total content of i-olefins, n-olefins, i-paraffins and n-paraffins of at least 40 wt.-%, more preferably at least 50 wt.-%, at least 55 wt.-%, at least 60 wt.-%, at least 65 wt.-% or at least 70 wt.-%. For example, the liquefied waste plastic feed may have a total content of i-olefins, n-olefins, i-paraffins and n-paraffins of 40 wt.-% to 100 wt.-%, such as 50 wt.-% to 100 wt.-%, 55 wt.-% to 100 wt.-%, 60 wt.-% to 100 wt.-%, 65 wt.-% to 100 wt.-%, 70 wt.-% to 100 wt.-%, such as 40 wt.-% to 99 wt.-%, 50 wt.-% to 99 wt.-%, 55 wt.-% to 99 wt.-%, 60 wt.-% to 99 wt.-%, 65 wt.-% to 99 wt.-%, 70 wt.-% to 99 wt.-%, or 70 wt.-% to 95 wt.-%.

In the present invention, a “total content” of multiple components refers to the summed content of each individual component. Accordingly, the liquefied waste plastic feed having a total content of i-olefins, n-olefins, i-paraffins and n-paraffins being at least 40 wt.-% means that the summed content, by weight, of i-olefins, n-olefins, i-paraffins and n-paraffins relative to the total weight of the liquefied waste plastic feed is 40 wt.-% or more, i.e. in the range of from 40 wt.-% to 100 wt.-%.

In the present invention, i-paraffins and n-paraffins are collectively designated as paraffins and thus the content of paraffins corresponds to the total content of i-paraffins and n-paraffins. In this respect, i-paraffins refer to branched non-cyclic alkanes, while n-paraffins refer to linear non-cyclic alkanes. In the present specification, i-paraffins may also be referred to as iso-paraffins and n-paraffins may also be referred to as normal-paraffins.

It is specifically preferred for the LWP feed to contain a high share of paraffins and olefins, since these materials are easy to crack. Aromatics, on the other hand, have a very low cracking tendency and thus do not contribute to the desired high-value products, in particular ethylene. It may even be favourable to remove excessive aromatics, in particular benzene, from the LWP feed or from the cracking product, in particular if the non-olefins part thereof is intended for fuel purposes due to regulatory restrictions of benzene content. In the present invention, aromatics refer to compounds having at least one aromatic ring, including condensed aromatics.

In addition, naphthenes are difficult to crack into valuable material as well and furthermore show a rather high coking tendency. Therefore, the higher the content of i-paraffins, n-paraffins, i-olefins and n-olefins in the LWP feed, the higher the expected yield of valuable products, such as ethylene and propylene, in particular ethylene.

In accordance with the present invention, naphthenes refer to cyclic non-aromatic branched or non-branched alkanes, alkenes or alkynes, including multiple unsaturated. Paraffinic naphthenes refer to cyclic branched or non-branched alkanes. Olefinic naphthenes refer to cyclic non-aromatic branched or non-branched alkenes, including multiple unsaturated.

The liquefied waste plastic feed typically has a total olefins content of 10 wt.-% or more, such as 15 wt.-% or more, 20 wt.-% or more or 30 wt.-% or more. Although containing a high amount of olefins is not as such favourable for the downstream process, LWP originally has a quite high content of olefins. Actually, as regards the LWP production process, a high olefins content is an indicator of a more effective depolymerisation process, eventually resulting in improved overall yield of valuable material. Surprisingly, the present invention's method, in particular the thermal cracking step, can handle such a challenging feed. In other words, it is neither necessary nor desirable that the olefins content be significantly reduced by specific measures such as hydrogenation, including saturation of olefins or total hydrogenation. As a matter of course, however, mild or selective hydrogenation is not excluded by the present invention. Such mild and/or selective hydrogenation may for example be used to convert diolefins, in particular conjugated diolefins, to monoolefins. Such mild and/or selective hydrogenation may similarly be used to convert alkynes to alkenes and/or alkanes and/or to convert MAPD, i.e. methyl acetylene and propadiene, to simple monoolefins. Mild hydrogenation is also useful for metal and halogen removal. Such mild and/or selective hydrogenation is preferred in particular because the aforementioned materials were found to be strong coke promotors.

Nevertheless, it is preferred for the liquefied waste plastic feed to have a total olefins content of 90 wt.-% or less, preferably 80 wt.-% or less, 70 wt.-% or less or 60 wt.-% or less. For example, the liquefied waste plastic feed may have a total olefins content in the range of 10 wt.-% to 90 wt.-%, such as 15 wt.-% to 80 wt.-%, 20 wt.-% to 70 wt.-%, 20 wt.-% to 60 wt.-%, or 30 wt.-% to 60 wt.-%.

The liquefied waste plastic feed preferably has a content ratio of monoolefins to diolefins of 10 or more by weight, preferably 12 or more, 15 or more or 20 or more. Although there is no specific upper limit, the content ratio may be for example 1000 or less, such as 100 or less. The inventors surprisingly found that a high relative content of monoolefins relative to diolefins significantly reduces the coking tendency. Specifically, while it was conventionally assumed that olefins in general result in high coking tendency, the inventors found that olefins mainly tend to produce high coke amounts if a high share of diolefins is present, and in particular conjugated diolefins result in high coking tendency.

The liquefied waste plastic feed of the present invention preferably has a content ratio of i-paraffins to n-paraffins of 0 to 2.0, preferably 1.7 or less, 1.5 or less, 1.0 or less, or 0.90 or less. Employing such a LWP feed having a rather low share of i-paraffins, in addition to having a i-olefin/n-olefin ratio as defined in claim 1, can further improve the ethylene yield of the cracking step. The content ratio of i-paraffins to n-paraffins may particularly be 0.80 or less, such as 0.70 or less, 0.60 or less, 0.50 or less, 0.40 or less, 0.30 or less, 0.20 or less, 0.15 or less, 0.10 or less, 0.09 or less, 0.08 or less, 0.07 or less, 0.06 or less, 0.05 or less, or 0.04 or less.

The liquefied waste plastic feed preferably has a total paraffins content of 5 wt.-% or more, 10 wt.-% or more, 12 wt.-% or more, 15 wt.-% or more, 17 wt.-% or more, 19 wt.-% or more, 20 wt.-% or more. The total paraffins content may for example be 5 wt.-% to 100 wt.-%, such as 5 wt.-% to 80 wt.-%, 5 wt.-% to 60 wt.-%, or 20 wt.-% to 60 wt.-%. A high content of paraffins can favourably increase ethylene yield, whereas aromatics are difficult to be converted to light olefins by thermal cracking. Thus, the liquefied waste plastic feed preferably has a total aromatics content of 0 wt.-% to 35 wt.-%, more preferably 30 wt.-% or less, 25 wt.-% or less, 20 wt.-% or less, or 15 wt.-% or less. The liquefied waste plastic feed preferably has a total monoaromatics content of 0 wt.-% to 30 wt.-%, more preferably 25 wt.-% or less, 20 wt.-% or less, or 15 wt.-% or less. Monoaromatics refer to compounds having exactly one aromatic ring, i.e. not including condensed aromatic rings or multiple non-condensed aromatic rings.

The liquefied waste plastic feed preferably has a total content of n-paraffins and n-olefins of 20 wt.-% to 100 wt.-%, more preferably 23 wt.-% or more, 25 wt.-% or more, 27 wt.-% or more, 30 wt.-% or more, such as 30 wt.-% to 100 wt.-% or 30 wt.-% to 95 wt.-%. The liquefied waste plastic feed preferably has a content ratio, by weight, of total n-paraffins and n-olefins to total i-paraffins and i-olefins ([nP]+[nO])/([iP]+[iO]) of 0.40 or more, more preferably 0.50 or more, 0.60 or more, 0.70 or more, 0.80 or more, 0.90 or more, 1.00 or more, 1.20 or more, 1.40 or more, 1.60 or more, 1.80 or more, 2.00 or more, 2.50 or more, or 3.00 or more. The content ratio ([nP]+[nO])/([iP]+[iO]) is not particularly limited and but may e.g. be 0.40 to 1000.00, such as 0.40 to 100.00, or 0.40 to 50.00. The liquefied waste plastic feed preferably has a total content of n-olefins, i-olefins and diolefins ([nO]+[iO]+[diO]) of 60 wt.-% or less, more preferably 55 wt.-% or less, or 50 wt.-% or less, such as 0 wt.-% to 60 wt.-%, 5 wt.-% to 60 wt.-%, or 10 wt.-% to 60 wt.-%. The liquefied waste plastic feed preferably has a content ratio of total n-olefins and i-olefins to diolefins (([nO]+[iO])/[diO]) of 5 or more, more preferably 8 or more, or 10 or more. The above ranges further improve the yield of favourable products.

Regarding the LWP feed, the following equation is preferably fulfilled:

$$[ iO ] * [ diO ] < α$$

wherein [iO] is the content by weight of i-olefins in the LWP feed and [diO] is the content by weight of diolefins in the LWP feed, and α is 100, preferably 90, 80, or 75.

Regarding the LWP feed, the following equation is preferably fulfilled:

$$( [ iO ] + 2 * [ iP ] ) * [ diO ] < β$$

wherein [iO] is the content by weight of i-olefins in the LWP feed and [diO] is the content by weight of diolefins in the LWP feed, and β is 150, preferably 130, 120, 100, 90, 80, or 75.

The present inventors surprisingly found that a high content of i-olefins and/or of total isomers in combination with diolefins, and especially conjugated diolefins, results in high coking tendency, even if the absolute amount of diolefins is rather low.

Preferably, the liquefied waste plastic feed has a content of diolefins of 0 wt.-% to 5.0 wt.-%, more preferably 4.0 wt.-% or less, 3.0 wt.-% or less, 2.5 wt.-% or less, 2.0 wt.-% or less, 1.5 wt.-% or less or 1.0 wt.-% or less. Preferably, the liquefied waste plastic feed has a content of conjugated diolefins of 0 wt.-% to 5.0 wt.-%, more preferably 4.0 wt.-% or less, 3.0 wt.-% or less, 2.0 wt.-% or less, 1.0 wt.-% or less, 0.75 wt.-% or less or 0.50 wt.-% or less.

It is also preferred for the liquefied waste plastic feed to have an acidity of 0 to 10.0 mgKOH/g, more preferably 8.0 mgKOH/g or less, 6.0 mgKOH/g or less, 4.0 mgKOH/g or less, 2.0 mgKOH/g or less, 1.5 mgKOH/g or less, 1.0 mgKOH/g or less, 0.9 mgKOH/g or less, 0.8 mgKOH/g or less, 0.7 mgKOH/g or less, 0.6 mgKOH/g or less, or 0.5 mgKOH/g or less. The acidity may be measured in accordance with ISO6619. The liquefied waste plastic feed may have an oxygen content of 0 wt.-% to 1.50 wt.-%, preferably 1.40 wt.-% or less, 1.30 wt.-% or less, 1.20 wt.-% or less, 1.10 wt.-% or less, 1.00 wt.-% or less, 0.90 wt.-% or less, or 0.80 wt.-% or less. Oxygen content in wt.-% can be determined by difference using the formula 100 wt.-%−(CHN content+ash content), wherein CHN content refers to combined content of carbon, hydrogen and nitrogen, as determined in accordance with ASTM D5291, and ash content refers to ash content as determined in accordance with ASTM D482/EN15403. Isoparaffins and i-olefins are more vulnerable to oxidation reactions than non-branched hydrocarbons, therefor it is beneficial to have as low oxygen content as possible in the LWP feed.

The liquefied waste plastic feed preferably has a total content of i-paraffins and i-olefins of 0 wt.-% to 50 wt.-%, more preferably 40 wt.-% or less, 30 wt.-% or less, or 20 wt.-% or less.

The liquefied waste plastic feed preferably has a content of C9 i-olefins of 0 wt.-% to 10 wt.-%, preferably 8 wt.-% or less, 6 wt.-% or less or 5 wt.-% or less. The inventors surprisingly found that specifically C9 i-olefins tend to significantly reduce the yield of ethylene in the cracking step.

The liquefied waste plastic feed preferably has a content of n-olefins of 2 wt.-% or more, more preferably 5 wt.-% or more, 10 wt.-% or more, 15 wt.-% or more, 20 wt.-% or more, or 25 wt.-% or more.

The liquefied waste plastic feed preferably has a total aromatics content of 35 wt.-% or less, more preferably 30 wt.-% or less, 25 wt.-% or less, 20 wt.-% or less, or 15 wt.-% or less.

Preferably, a fraction of the liquefied waste plastic feed having a T95 temperature of 180° C. or less, preferably 165° C. or less, has a total content of C9 i-olefins of 10 wt.-% or less, preferably 8 wt.-% or less, 6 wt.-% or less or 5 wt.-% or less. The T95 temperature (95 vol-% recovered) is determined in accordance with EN ISO 3405-2019.

Preferably, the liquefied waste plastic feed essentially consists of liquid products of a waste plastic liquefaction process, or a fraction thereof. In other words, the liquefied waste plastic feed (LWP feed) preferably contains only small or negligible amounts of gaseous (NTP) components and solids/non-evaporating material, such as char. Depending on the LWP feed production method, the effluent of a waste plastic liquefaction process may contain large amounts of gaseous (NTP) components, in some cases even exceeding 50 wt.-% of the effluent, i.e. of the liquefaction product. The term “consisting essentially of” herein refers to a content of at least 95 wt.-% and may be up to 100 wt.-%.

Specifically, the liquefied waste plastic feed preferably has a content of gaseous (NTP) components of 1.5 wt.-% or less, preferably 1.0 wt.-% or less, more preferably 0.5 wt.-% or less. The content of gaseous (NTP) components may for example be 0 wt.-% to 1.5 wt.-%. Herein, gaseous (NTP) components refer to components being gaseous at normal pressure of 101.325 kPa absolute and temperature of 20° C. (NTP). Such gaseous (NTP) components typically contain high share of light olefins, in particular ethylene, propylene, butene(s) and butadiene(s), which can better be utilized/separated before cracking. Furthermore, hydrocarbons in the gaseous (NTP) components tend to have very different optimal thermal cracking conditions compared to the non-gaseous (NTP) components so that cracking gaseous (NTP) components together with non-gaseous (NTP) components is not favourable.

Similarly, the liquefied waste plastic feed preferably has a content of solid components of 1000 wt.-ppm or less, preferably 100 wt.-ppm or less, more preferably 10 wt.-ppm or less, since such components may cause problems in the cracking stage. The solid components may be absent (content is 0 wt.-ppm). In the present invention, solid components refer to components which are insoluble in the LWP feed. Their content can thus be determined in accordance with ISO 663. Examples of solid components in the LWP feed include coke particles coming from heating tubes, iron scales from corrosion, dissolved impurities such as iron-, arsenic-, or calcium-containing compounds, sodium chloride, silicon contained in upstream additives, and other impurities form upstream processes.

Solid components may be abundant in liquefied waste plastic. Thus, it is preferred that the LWP or a fraction thereof be pre-treated to provide the LWP feed, wherein the pre-treatment comprises at least removal of solid components. Conventional techniques and equipment may be employed for removing solid components. Such equipment include desalters, filters, coalescers, etc. Such equipment may for example be provided in the feed train of the thermal cracking apparatus. Thus a low solids content in the feed entering the thermal cracking apparatus can be ensured.

The liquefied waste plastic feed may be an optionally pre-treated waste plastic pyrolysis oil (WPPO) or an optionally pre-treated fraction thereof, or an optionally pre-treated waste plastic-based hydrothermal liquefaction oil (HTL oil) or an optionally pre-treated fraction thereof. The term waste plastic-based HTL oil refers to an oil produced by hydrothermal liquefaction of waste plastic.

The liquefied waste plastic feed preferably comprises an optionally pre-treated fraction of products of waste plastic liquefaction, preferably of liquefaction of polyolefin rich waste plastic, more preferably of liquefaction of polyethylene rich waste plastic. In the context of the present invention, the term “polyolefin-rich” refers to a polyolefins content relative to the whole waste plastic being subjected to liquefaction of 50 wt.-% to 100 wt.-%, preferably at least 60 wt.-% or at least 70 wt.-%. Similarly, the term “polyethylene-rich” refers to a polyethylene content relative to the whole waste plastic being subjected to liquefaction of 50 wt.-% to 100 wt.-%, preferably at least 60 wt.-% or at least 70 wt.-%. The content of polyolefins and/or polyethylene may be determined using any commonly known method, such as DSC, melting enthalpy, FT-IR, NMR, in particular solid state NMR, which are usual in laboratories.

More specifically, the DSC melting enthalpy at 70-130° C., E_{70-130° C.}, and the DSC melting enthalpy at 130-180° C., E_{130-180° C.}, may be used to determine and differentiate between polypropylene and polyethylene. DSC, i.e. dynamic scanning calorimetry, may be conducted under conditions of 5° C. temperature rise per minute and is preferably measured on a homogenised sample of the solid waste plastic to be subjected to depolymerisation. In this respect, the DSC melting enthalpy at 70-130° C. corresponds to polyethylene material in the waste plastic and the DSC melting enthalpy at 130-180° C. corresponds to polypropylene material in the waste plastic.

FT-IR peaks at 1168 cm⁻¹ and 716 cm⁻¹ may also be used to determine the relative content of polyethylene-based material.

In the context of the present invention, however, a determination of the content as commonly applied in industrial waste plastic sorting plants, namely based on near-IR (NIR) analysis, is fully sufficient and preferably applied in the present invention. Employing a high share of polyolefins, in particular polyethylene, favourably influences the composition of the LWP feed and in particular increases the ethylene yield in the thermal cracking step.

The liquefied waste plastic feed preferably comprises or consists of a naphtha fraction of liquefied waste plastic or a middle distillate fraction of liquefied waste plastic. It particularly preferably comprises or consists of a naphtha fraction of liquefied waste plastic. The present inventors surprisingly found that a naphtha fraction of LWP causes considerably less coking.

The step of providing the liquefied waste plastic feed may comprise a stage of separating at least gaseous (NTP) components from liquefied waste plastic, i.e. separating at least components that are gaseous at normal pressure and temperature (“NTP”). The stage of separating at least gaseous (NTP) components may simply be referred to as degassing. The result of degassing may be referred to as degassed liquefied waste plastic. Thus, preferably, the above-mentioned low content of gaseous (NTP) components can be achieved. The step of providing the liquefied waste plastic feed may specifically comprise a stage of separating at least gaseous (NTP) components from liquefied waste plastic and recovering at least olefins from the gaseous (NTP) components. Thus, the light olefins in the gaseous (NTP) components, which are valuable materials, can be added to the value chain rather than being subjected to the thermal cracking step, thus improving yield, in particular ethylene yield, of the overall process.

The step of providing the liquefied waste plastic feed may comprise a stage of fractionating liquefied waste plastic, such as crude LWP, i.e. the optionally degassed effluent of a liquefaction process, thus providing a fraction of LWP. The fractionation stage preferably yields a middle distillate fraction and/or a naphtha fraction, more preferably a naphtha fraction. Each of these fractions can then be provided as the liquefied waste plastic feed of the present invention, after optional pre-treatment, and subjected to the cracking step.

The step of providing the liquefied waste plastic feed preferably comprises pre-treating liquefied waste plastic or a fraction of liquefied waste plastic. Suitable pre-treatments may be carried out before and/or after fractionation. For ease of reference, the optionally pre-treated liquefied waste plastic may simply be referred to as liquefied waste plastic and the optionally pre-treated fraction of liquefied waste plastic may simply be referred to as fraction of liquefied waste plastic.

The step of providing the liquefied waste plastic feed may comprise a stage of separating high-boiling components from liquefied waste plastic. The “high-boiling” components, or simply “heavies”, are preferably components boiling above 450° C. at normal pressure. Depending on the used liquefying conversion technology, the LWP may or may not contain heavies. If substantial amounts are present e.g. in the crude LWP, the heavies are preferably removed before the thermal cracking stage since they do not that easily convert to ethylene and/or propylene. Alternatively, the liquefying conversion technique may be selected such that potential heavies are processed and thus do not end up in the liquefying conversion effluent, i.e., in the crude LWP.

The step of providing the liquefied waste plastic feed may comprise a stage of thermally depolymerizing waste plastic. That is, thermal depolymerisation is a standard method for producing LWP. Among thermal polymerisation methods, hydrothermal liquefaction (HTL) and pyrolysis, such as fast pyrolysis, may be mentioned.

The liquefied waste plastic feed may be based on sorted waste plastic. In other words, the solid waste plastic, i.e. the waste plastic before being subjected to liquefaction, may comprise, essentially consist of or consist of sorted waste plastic. In particular in consumer plastic recycling, sorted grades of waste plastic are readily available. Thus, the composition of the solid waste plastic, and consequently the composition and/or properties of the LWP feed, can be adjusted not only using varying liquefaction conditions but similarly using different solid waste plastic grades, such as different grades of sorted waste plastic.

The solid waste plastic may also contain a mixture of different species of sorted waste plastic. Similarly, the liquefied waste plastic may comprise different species of optionally pre-treated liquefied sorted waste plastic. The liquefied waste plastic feed may comprise, essentially consist of or consist of mixtures of different fractions of optionally pre-treated liquefied waste plastic and/or mixtures of fractions of different species of optionally pre-treated liquefied sorted waste plastic.

The solid waste plastic particularly preferably comprises polyolefin-rich waste plastic, more preferably polyethylene-rich waste plastic. The polyethylene (PE) may be any kind of polyethylene such as LLDPE (linear low density PE), LDPE (low density PE) or HDPE (high density PE). Polyolefin-rich waste plastic is preferable as a solid waste plastic from which the LWP feed may be derived because it can be more easily converted into LWP feed having the favourable total content of i-olefins, n-olefins, i-paraffins and n-paraffins as well as i-olefins to n-olefins ratio. Further, such a material tends to have lower content of diolefins. Moreover, high polyolefin content in the solid waste plastic tends to result in lower oxygenates content in the LWP feed, contributing in particular to low carbon monoxide formation during thermal cracking, thus favouring the especially desired ethylene manufacturing and reducing down-time due to less impurity species known to be harmful for polymerization catalysts. Polyethylene-rich solid waste plastic is more preferred as facilitating even lower i-olefins to n-olefins ratio. Further, the total isomer content, i.e. the summed content of i-olefins and i-paraffins, can be adjusted to be lower which contributing to less coke formation during thermal cracking, which is presumed to be related to reduced MAPD formation. Specifically, it is presumed that high MAPD content in the product indicates that there may have been high levels of the unstable intermediate cyclopropyne (C₃H₂) or cyclopropene (C₃H₄). These intermediates are particularly strong coke promoters and rapidly contribute to polyaromatic formation.

The step of providing the liquefied waste plastic feed may comprise a stage of depolymerizing solid waste plastic to provide liquefied waste plastic.

The step of providing the liquefied waste plastic feed may further comprise a stage of pre-processing solid waste plastic. The pre-processing stage may comprise separating the solid waste plastic from other waste, such as paper, metal and/or compostable waste. The pre-processing stage may comprise washing the solid waste plastic with water. The pre-processing stage may comprise drying the solid waste plastic. The pre-processing stage may comprise reducing the size of the solid waste plastic, such as cutting the solid waste plastic, shredding the solid waste plastic and/or comminuting the solid waste plastic. The pre-processing stage may comprise melting the solid waste plastic. In this respect, melting means converting the solid waste plastic in a melt essentially without decomposing the polymer backbone.

The stage of depolymerizing solid waste plastic may comprise thermal depolymerisation to provide liquefied waste plastic. The stage of depolymerizing solid waste plastic may comprise hydrothermal liquefaction to provide waste plastic hydrothermal liquefaction oil (HTL oil). The stage of depolymerizing solid waste plastic may comprise thermal pyrolysis to provide waste plastic pyrolysis oil (WPPO), wherein the thermal pyrolysis may be thermal non-catalytic pyrolysis or thermal catalytic pyrolysis. In the present specification, the waste plastic hydrothermal liquefaction oil and the waste plastic pyrolysis oil are both embodiments of a fraction of liquefied waste plastic, more specifically a liquid (NTP) fraction thereof.

Specific conditions of the depolymerisation processes usable in the present invention are set forth below.

In general, both hydrothermal liquefaction and pyrolysis are thermal liquefaction processes.

In hydrothermal liquefaction (HTL) of waste plastic, the waste plastic is contacted with supercritical water, or sub- or near-critical water, typically at temperature from 250° C. to 550° C., preferably 280° C. to 400° C.; typically at a pressure from 5 MPa to 50 MPa absolute, preferably 7 MPa to 30 MPa; contact time typically being 0.1 h to 10 h, preferably 0.5 h to 6 h. Hydrothermal liquefaction is typically carried out under non-oxidative conditions. Non-oxidative conditions may be achieved for example by purging the liquefaction equipment with an inert gas such as nitrogen. By lowering the temperature and the contact time less gaseous (NTP) and more liquid (NTP) products may be obtained.

Typically, hydrothermal liquefaction is more tolerant towards moisture in the waste plastics than other liquefaction methods. Therefore, waste plastics for HTL would require less drying efforts than for pyrolysis. Overall, HTL may thus achieve liquefaction with less energy consumption. Moreover, in HTL, that hydrogen-ions, which tend to be released in the course of the process, may enhance heteroatom cleavage and stabilize coke-forming radicals. Thus, HTL may provide a liquefied waste plastic containing less heteroatoms. Moreover, LWP derived from a HTL process has such a quality that there may be less coking in the down-stream processing, such as thermal cracking and in particular steam cracking.

Thermal non-catalytic pyrolysis typically employs a temperature in the range from 300° C. to 850° C., preferably 400° C. to 800° C. This process is typically conducted at atmospheric pressure, usually under non-oxidative conditions, especially in the absence of air. The non-oxidative conditions may be achieved for example by purging the liquefaction equipment with an inert gas such as nitrogen.

Thermal catalytic pyrolysis typically employs a temperature in the range from 250° C. to 500° C., preferably from 300° C. to 450° C. This process is typically conducted at atmospheric pressure, usually under non-oxidative conditions, especially in the absence of air. The process typically employs a solid catalyst, preferably an acidic solid catalyst, for example an acidic FCC catalyst, an acidic zeolite catalyst or an acidic silica-alumina catalyst, such as ZSM-5 or H-ultrastable Y-zeolite, just to name a few. The non-oxidative conditions may be achieved for example by purging the liquefaction equipment with an inert gas such as nitrogen.

The liquefied waste plastic feed preferably comprises optionally pre-treated depolymerized solid waste plastic or a fraction thereof, preferably optionally pre-treated thermally depolymerized waste plastic or a fraction thereof. The solid waste plastic preferably comprises sorted waste plastic. The solid waste plastic may comprise polyolefin-rich waste plastic. The solid waste plastic may specifically comprise polyethylene-rich waste plastic.

The solid waste plastic is preferred to have an oxygen content of 15 wt.-% or less, preferably 10 wt.-% or less, more preferably 5 wt.-% or less, of the total weight of the solid waste plastic. Oxygen content in wt.-% can be determined by difference using the formula 100 wt.-%−(CHN content+ash content), wherein CHN content refers to combined content of carbon, hydrogen and nitrogen, as determined in accordance with ASTM D5291, and ash content refers to ash content as determined in accordance with ASTM D482/EN15403.

The solid waste plastic preferably comprises waste plastic, in which the ratio between the melting enthalpy in the temperature range of 70-130° C. and the melting enthalpy in the temperature range of 130-180° C., E_{70-130° C.}/E_{130-180° C.}, as measured by DSC, is 50% to 100%, preferably 60% or more or 70% or more.

Similarly, the solid waste plastic preferably comprises waste plastic, in which the intensity ratio between the intensity I₁₁₆₈ of the FT-IR peak at 1168 cm⁻¹, and the total intensities I₁₁₆₈+I₇₁₆ of peaks at 1168 cm⁻¹ and 716 cm⁻¹, I₁₁₆₈/[I₁₁₆₈+I₇₁₆], is 0 to 0.3, preferably 0.2 or less.

The intensity ratio I₁₁₆₈/[I₁₁₆₈+I₇₁₆] was found to be particularly useful when determining the content of polyethylene in a waste plastic material (cf. Larsen et al, “Determining the PE fraction in recycled PP”, Polymer testing, vol. 96, April 2021, 107058) and a ratio of 0.3 or less is considered to be favourable in the present invention. The article by Larsen et al furthermore describes the FT-IR method usable in the present invention and the DSC method for determining melting enthalpy in accordance with the present invention.

The liquefied waste plastic feed preferably has an initial boiling point of 150° C. or less, preferably 120° C. or less or 100° C. or less.

Preferably, the liquefied waste plastic feed is obtained by liquefaction of solid waste plastic, followed by gas-liquid separation and optional fractionation as well as optional further pre-treatment(s). The liquefied waste plastic feed may comprise or consist of any fraction of liquefied waste plastics (LWP), preferably a degassed fraction of the liquefaction process effluent, or a liquid and/or waxy fraction of liquefied waste plastic, or a mixture of two or more thereof. The waxy fraction of LWP may sometimes also referred to as a tetrahydrofuran-extractable LWP fraction. In particular, the LWP feed may comprise or consist of a naphtha fraction and/or middle distillate (MD) fraction of LWP. As said before, any of the fractions mentioned herein may be pre-treated before fractionation or after fractionation or before and after fractionation.

In the present invention, a naphtha fraction of LWP is preferably a fraction having a T5 temperature of 20° C. or more, preferably 25° C. or more, or 30° C. or more and a T95 temperature of 180° C. or less, more preferably having a T5-T95 boiling range of 30-180° C., 40-180° C., or 40-165° C. The difference between T5 temperature and T95 temperature (T95−T5) may be at least 20° C., such as in the range from 20° C. to 150° C., 20° C. to 120° C., or 30° C. to 80° C. A MD fraction of LWP is a fraction having a T5 temperature of 160° C. or more and a T95 temperature of 360° C. or less, more preferably having a T5-T95 boiling range of 160-360° C., 160-330° C., or 165-300° C. The difference between T5 temperature and T95 temperature may be at least 40° C., such as in the range from 40° C. to 200° C., 40° C. to 150° C., or 50° C. to 100° C. Boiling points and ranges refer to atmospheric pressure, i.e. normal pressure of 101.325 kPa, if not indicated to the contrary. The T5 temperature (5 vol-% recovered) and T95 temperature (95 vol-% recovered) as well as other such temperatures may be determined in accordance with EN ISO 3405-2019.

The thermal cracking in step (b) may comprise steam cracking. Since steam cracking is tolerant to a variety of impurities (especially compared to catalytic processes), this cracking technique is generally preferred in the present invention.

The feed to the thermal cracking reactor, also referred to as the total cracker feed, may comprise sulphur 20-300 ppm by weight, preferably 20-250 ppm by weight, more preferably 20-100 ppm by weight, and even more preferably 50-65 ppm by weight. The feed to the thermal cracking reactor refers to the liquefied waste plastic feed, optional co-feed(s) and optional additive(s).

Any conventional thermal cracking additive(s) may be added to cracking step (b). Examples of such conventional thermal cracking additives include sulphur containing species (sulphur additives), such as dimethyl disulphide (DMDS), or carbon disulphide (CS2). DMDS is a particularly preferred sulphur additive. Sulphur additive(s) may be mixed with the LWP feed, with optional co-feed(s) or with both, before feeding the LWP feed and the optional co-feed(s) to the thermal cracking. Alternatively, or in addition, sulphur additive(s) may be added by injecting into the thermal cracking furnace a diluent, preferably steam, comprising sulphur additive(s).

The thermal cracking step (b) may be conducted under a coil outlet temperature (COT) selected from the range from 750° C. to 920° C., preferably from 750° C. to 890° C., more preferably from 800° C. to 880° C., and even more preferably from 820° C. to 860° C.

When subjecting conventional feed to thermal cracking, high cracking temperatures tend to favour ethylene production. However, when employing a cracker feed comprising LWP feed of the present invention, it was surprisingly found that the same ethylene yield can be achieved at lower COT. It is assumed that this effect is at least partly associated with the high olefins content commonly present in LWP.

The thermal cracking step (b) may be conducted under conventional conditions such as a coil outlet pressures (COP) selected from a range from 1.3 bar to 6.0 bar, preferably from 1.3 bar to 3.0 bar. If not specified otherwise, pressure relates to absolute pressure.

When employing a thermal cracking diluent, such as steam in steam cracking, the thermal cracking step (b) is preferably conducted at a flow rate ratio between thermal cracking diluent, preferably steam, and LWP feed and optional co-feed(s) (flow rate of diluent [kg/h]/flow rate of LWP feed and optional co-feed(s) [kg/h]) within a range from 0.10 to 1.00, preferably from 0.25 to 0.85.

The step (c) may comprise recovering a recycle stream from the thermal cracking effluent and, after one or more optional hydrotreatment(s) and/or further post-processing, subjecting the recycle stream to thermal cracking in the thermal cracking step (b) as a co-feed with the LWP feed. Recycling unconverted reactants increases the overall profitability and the overall yield of the thermal cracking process and/or the overall yield of the desired products such as ethylene and propylene, especially of ethylene. The one or more optional hydrotreatment(s) may specifically comprise mild or selective hydrogenation of diolefins, optionally followed by full hydrogenation to alkane(s).

The step (c) may further comprise separating other fraction(s) from the effluent of the thermal cracking reactor. Preferably, the step (c) comprises separating at least a fraction comprising propylene from the effluent of the thermal cracking reactor. Other fraction(s) may be separated as well, such as a fraction comprising at least butene, a fraction comprising at least ethane, a fraction comprising at least propane, a fraction comprising at least butane, a fraction comprising at least hydrogen. These fractions may be product fractions of the present method. These fractions may be separated in a single stage or may be separated in multiple subsequent stages. Preferably, a fraction rich in C2 hydrocarbons is separated and this fraction is then further separated at least into a fraction comprising ethene and a fraction comprising ethane. Such separation of a fraction rich C2 hydrocarbons, e.g. a fraction comprising 30 wt.-% to 100 wt.-%, preferably at least 40 wt.-% C2 hydrocarbons, may be forwarded to a C2 splitter to provide a fraction comprising ethene and a fraction comprising ethane. Similarly, a fraction rich in C3 hydrocarbons may be separated and this fraction is then further separated at least into a fraction comprising propene and a fraction comprising propane. Such separation of a fraction rich C3 hydrocarbons, e.g. a fraction comprising 30 wt.-% to 100 wt.-%, preferably at least 40 wt.-% C3 hydrocarbons, may be forwarded to a C3 splitter to provide a fraction comprising propene and a fraction comprising propane. The fraction rich C3 hydrocarbons may be separated after the fraction rich C2 hydrocarbons has been separated or may be separated in the same stage. Each of these fraction may be recovered as a product fraction of the method or may be further purified or post-processes to give a product fraction of the method.

The step (a) of providing the liquefied waste plastic feed may comprise one or more optional pre-treatment stages. A pre-treatment stage is a stage in which liquefied waste plastic or a fraction thereof is subjected to a pre-treatment.

The pre-treatment may comprise drying the liquefied waste plastic. Drying may be accomplished by conventional means, such as phase separation, evaporation, and/or moisture absorbents.

The pre-treatment may comprise separation of solid particles from the liquefied waste plastic feed. Such solid particles may comprise char or other solids. Separation of solid particles may be accomplished by conventional means such as settling, centrifugation, decanting and/or filtering. Settling may include gravity settling and/or induced settling.

The pre-treatment may comprise separation of gaseous (NTP) components from the liquefied waste plastic feed. Such separation may be accomplished by known means, such gas-liquid separation and/or fractionation.

The pre-treatment may comprise selective hydrogenation and/or mild hydrogenation of the liquefied waste plastic feed. Selective and/or mild hydrogenation may encompass selective hydrogenation of dienes and/or alkynes to monoolefins or alkanes, mild hydrogenation essentially converting dienes and/or alkynes to monoolefins or alkanes but preferably essentially not converting monoolefins to alkanes. Preferably, the pre-treatment essentially not includes converting monoolefins to alkanes and particularly preferably does not include full hydrogenation of monoolefins to alkanes. Selective and/or mild hydrogenation may particularly encompass selective hydrogenation of MAPD to monoolefins or alkanes, mild hydrogenation essentially converting MAPD to monoolefins or alkanes but preferably essentially not converting monoolefins to alkanes.

The method may comprise providing multiple thermal cracker furnaces, and performing thermal cracking step (b) in at least one of the multiple thermal cracker furnaces. Specifically, it is possible to obtain cracking products from the multiple thermal cracking furnaces, mixing the obtained thermal cracking effluents to form a combined cracking product, and optionally subjecting at least a portion of the combined cracking product to a purification treatment to remove at least one of CO, CO₂, MAPD, and C₂H₂. Of these, MAPD and acetylene may subsequently be converted to valuable products such as ethylene and propylene.

The thermal cracking in step (b) may be carried out in the presence of a co-feed. The presence of a co-feed(s) may be used to lower the diolefin-content of the total cracker feed. The co-feed may also serve to reduce halogens content of the steam cracker feed (total cracker feed) in case the LWP feed has high halogens content (which is usual in post-consumer waste plastic). Finally, adding a co-feed, such as a conventional steam cracker feed (e.g. a conventional naphtha feed), may reduce coking tendency while the benefits of the present invention are still maintained. In this respect, the total cracker feed refers to the material which is fed to the thermal cracking step (b) except for cracking diluent, and thus the total cracker feed comprises at least the LWP feed and may further comprise at least one co-feed and/or at least one additive. If the diolefins content is, however, low in the LWP feed, less of co-feed is needed for “diluting” the LWP feed before cracking. The co-feed is preferably such that the properties of the total cracker feed are shifted towards more preferred values of the above-mentioned properties of the LWP feed by addition of the co-feed(s).

The co-feed may be a hydrocarbon feed, in particular a fossil hydrocarbon feed. The term fossil may also be referred to as crude oil-derived. The co-feed is preferably a fossil naphtha feed. The fossil naphtha feed may for example be full range naphtha or light naphtha. In accordance with the present invention, naphtha preferably has an initial boiling point of at least 30° C., such as in the range of 30° C. to 80° C., and a final boiling point of at most 200° C., such as in the range of 100° C. to 200° C. Full range naphtha has an initial boiling point of at least 35° C. and a final boiling point of at most 180° C. Light naphtha has an initial boiling point of at least 30° C. and a final boiling point of at most 120° C.

The at least one co-feed may specifically be a fossil naphtha feed fulfilling the following open naphtha specification “Platts specification guide to refined oil products” (in its version of September 2021), as available from https://www.spglobal.com/platts/PlattsContent/_assets/_files/en/our-methodology/methodology-specifications/europe-africa-refined-products-methodology.pdf
Specific gravity at 15° C. is at most 0.735 g/ml,
Reid Vapour Pressure is at most 12.5 psi,
Colour is at least +20 Saybolt,
Initial Boiling point is at least 30° C.,
Final Boiling point is at most 180° C.,
Paraffins content is at least 65% by volume, Olefins content is at most 1% by volume, the balance being naphthenes and aromates,
Sulphur content is at most 500 ppm,
H2S content is at most 10 ppm,
Lead content is at most 50 ppb,
content of organic chlorides is at most 3 ppm,
MTBE content is at most 50 ppm,
Total oxygenates content is at most 100 ppm,
Mercury content is at most 5 ppb.

The content of the LWP feed in the total cracker feed is preferably at least 2 wt.-%, more preferably at least 5 wt.-%, at least 10 wt.-%, at least 15 wt.-%, at least 20 wt.-%, at least 25 wt.-%, at least 30 wt.-%, at least 35 wt.-%, at least 40 wt.-%, at least 45 wt.-%, at least 50 wt.-%, at least 55 wt.-% or at least 60 wt.-%. The content of the LWP feed in the total cracker feed may for example be in the range of 2 wt.-% to 100 wt.-%, 5 wt.-% to 80 wt.-%, or 10 wt.-% to 70 wt.-%.

The thermal cracking in step (b) may preferably be carried out in the presence of a co-feed having a total content of aromatics and naphthenes of 0 wt.-% to 35 wt.-%, preferably at most 30 wt.-%, at most 25 wt.-%, at most 20 wt.-%, at most 15 wt.-% or at most 10 wt. %.

Further, the thermal cracking in step (b) may be carried out in the presence of a co-feed having an total olefins content of 0 wt.-% to 3.0 wt.-%, preferably at most 1.0 wt.-%, at most 0.75 wt.-%, at most 0.50 wt.-%, at most 0.30 wt.-% or at most 0.10 wt. % and/or having a total oxygenates content of 0 wt.-% to 1.0 wt.-%, preferably at most 0.50 wt.-%, at most 0.30 wt.-%, at most 0.10 wt. %, or at most 0.05 wt.-%.

The thermal cracking in step (b) may be carried out in the presence of renewable hydrocarbon co-feed, such as a HVO hydrocarbon co-feed. In the present invention, HVO hydrocarbons are obtainable by catalytic hydrotreatment of vegetable oils and/or animal fats yielding mainly paraffins.

The thermal cracking in step (b) may carried out in the presence of a Fischer-Tropsch hydrocarbon co-feed. Fischer-Tropsch based hydrocarbons are particularly preferable because they usually contain low amounts of aromatics, olefins, naphthenes and heteroatoms. Fischer-Tropsch hydrocarbons are obtainable by catalytic conversion of syngas comprising carbon monoxide and hydrogen, typically yielding a substantially Gaussian distribution of hydrocarbon chains, mainly n-paraffins. The Fischer-Tropsch based hydrocarbons may be of fossil nature, of renewable nature or both. Distinction between fossil and renewable can be made based on biogenic carbon content. Specifically, the renewable or fossil origin of any organic compounds, including hydrocarbons, can be determined by suitable method for analysing the content of carbon from renewable sources e.g. DIN 51637 (2014), ASTM D6866 (2020) and EN 16640 (2017). These methods are based on the fact that carbon atoms of renewable or biological origin comprise a higher number of unstable radiocarbon (¹⁴C) atoms compared to carbon atoms of fossil origin. Therefore, it is possible to distinguish between carbon compounds derived from renewable or biological sources or raw material and carbon compounds derived from fossil sources or raw material by analysing the ratio of ¹²C and ¹⁴C isotopes. Thus, a particular ratio of said isotopes can be used as a “tag” to identify a renewable carbon compound and differentiate it from non-renewable carbon compounds. The isotope ratio does not change in the course of chemical reactions. Therefore, the isotope ratio can be used for identifying renewable compounds, components, and compositions and distinguishing them from non-renewable, fossil materials in reactor feeds, reactor effluents, separated product fractions and various blends thereof. As used herein, the content of carbon from biological or renewable sources is expressed as the biogenic carbon content meaning the amount of biogenic carbon in the material as a weight percent of the total carbon (TC) in the material in accordance with ASTM D6866 (2020) or EN 16640 (2017).

Within step (c), the method of the present invention may comprise a stage of subjecting at least a portion of the thermal cracking effluent to a post-treatment (or purification treatment) to remove at least one of CO, CO₂, MAPD and C₂H₂. Other post-treatments, such as further fractionation of one or more effluent fraction, may be carried out as well.

The present invention furthermore provides a LWP feed for thermal cracking having a content ratio of i-olefins to n-olefins of 3.0 or less. The LWP feed preferably comprises an optionally pre-treated liquefied waste plastic or a fraction thereof, such as an optionally pre-treated waste plastic thermally depolymerized oil or a fraction thereof. In addition, the present invention provides a LWP feed for thermal cracking which is an optionally pre-treated liquefied waste plastic obtained from depolymerisation of polyethylene-rich waste plastic, or a fraction thereof.

The LWP feeds of the present invention preferably have one, more or all of the properties recited for the LWP feed being subjected to thermal cracking in step (b) of the method of the present invention.

### EXAMPLES

In the following, the present invention is demonstrated by means of non-limiting Examples. Nevertheless, it is to be understood that the Examples represent preferred embodiment of the invention and, in particular, numerical values and range recited in the Examples may be combined with other ranges and values disclosed in the specification to give new ranges embodying the invention.

In the following Examples and Comparative Examples, different LWP feeds were subjected to steam cracking as an embodiment of thermal cracking. The LWP feeds were analysed before cracking to determine their relative contents of n-paraffins, i-paraffins, n-olefins, i-olefins, di-olefins, naphthenes and aromatics using the PIONA method. This PIONA method may generally be used in the present invention to determine the composition of a material, such as a LWP feed.

The PIONA method is described by Pyl et al, Journal of Chromatography A, 1218 (2011) 3217-3223.

A feed derived from depolymerised sorted polyethylene-based waste plastic was degassed and fractionated into a middle distillate fraction (PE_MD; boiling range, IBP to FBP, about 160-350° C.) and a naphtha range fraction (PE_N; boiling range about 35-190° C.). The naphtha fraction and the middle distillate fraction were analysed by PIONA to give n-paraffins (nP), i-paraffins (iP), n-olefins (nO), i-olefins (iO), diolefins (diO), paraffinic naphthenes (pN), olefinic naphthenes (ON) and aromatics (A) contents per carbon number. The results (numbers rounded) are as follows:

TABLE 1

| Sample | C No | nP | iP | nO | iO | diO | pN | oN | A |
|---|---|---|---|---|---|---|---|---|---|
| PE_N | 5 | 0.2 | 0.0 | 0.2 | 0.0 | 0.0 | 0.0 | 0.0 | 0.0 |
| PE_N | 6 | 1.2 | 0.0 | 3.1 | 0.2 | 0.0 | 0.0 | 0.3 | 1.6 |
| PE_N | 7 | 4.0 | 0.0 | 5.7 | 0.8 | 0.0 | 0.2 | 2.1 | 4.1 |
| PE_N | 8 | 5.7 | 0.0 | 7.4 | 1.2 | 0.5 | 2.7 | 3.0 | 2.0 |
| PE_N | 9 | 5.3 | 0.4 | 7.6 | 2.0 | 0.4 | 3.5 | 4.3 | 3.3 |
| PE_N | 10 | 4.0 | 0.1 | 8.4 | 2.3 | 0.5 | 1.2 | 3.6 | 1.7 |
| PE_N | 11 | 0.5 | 0.1 | 1.1 | 0.1 | 0.2 | 0.9 | 1.9 | 0.1 |
| PE_N | 12 | 0.0 | 0.0 | 0.0 | 0.0 | 0.0 | 0.1 | 0.2 | 0.0 |
| PE_MD | 8 | 0.0 | 0.0 | 0.0 | 0.1 | 0.0 | 0.0 | 0.0 | 0.0 |
| PE_MD | 9 | 0.0 | 0.0 | 0.0 | 0.0 | 0.0 | 0.0 | 0.0 | 0.0 |
| PE_MD | 10 | 0.8 | 0.0 | 0.9 | 0.1 | 0.0 | 0.0 | 0.0 | 0.9 |
| PE_MD | 11 | 3.2 | 0.0 | 4.5 | 0.5 | 0.3 | 0.5 | 0.9 | 1.4 |
| PE_MD | 12 | 3.2 | 0.0 | 4.4 | 1.5 | 0.1 | 0.5 | 1.7 | 1.6 |
| PE_MD | 13 | 3.0 | 0.0 | 4.0 | 1.9 | 0.3 | 0.8 | 0.5 | 1.1 |
| PE_MD | 14 | 3.2 | 0.0 | 3.9 | 1.7 | 0.8 | 0.2 | 1.0 | 1.4 |
| PE_MD | 15 | 3.1 | 0.0 | 3.5 | 1.5 | 0.1 | 0.7 | 1.0 | 0.4 |
| PE_MD | 16 | 2.9 | 0.0 | 3.3 | 0.7 | 0.7 | 0.9 | 1.0 | 0.3 |
| PE_MD | 17 | 2.9 | 0.0 | 2.8 | 1.5 | 0.9 | 0.0 | 0.9 | 0.0 |
| PE_MD | 18 | 2.7 | 0.0 | 2.6 | 0.6 | 0.9 | 1.2 | 0.6 | 0.0 |
| PE_MD | 19 | 2.0 | 0.0 | 2.1 | 1.4 | 0.7 | 0.8 | 0.3 | 0.0 |
| PE_MD | 20 | 0.6 | 0.0 | 0.7 | 0.7 | 0.2 | 0.6 | 0.6 | 0.0 |
| PE_MD | 21 | 0.1 | 0.0 | 0.1 | 0.2 | 0.0 | 0.1 | 0.2 | 0.0 |
| PE_MD | 22 | 0.0 | 0.0 | 0.0 | 0.0 | 0.0 | 0.0 | 0.0 | 0.0 |

In the same manner, feeds having high content of isoparaffins were provided. These feeds were naphtha fraction (PP_N; boiling range about 35-190° C.) and a middle distillate fraction (PP_MD; boiling range about 160-350° C.) of a depolymerized sorted polypropylene material. The PIONA analyses of these fractions are shown below (numbers rounded).

TABLE 2

| Sample | C No | nP | iP | nO | iO | diO | pN | oN | A |
|---|---|---|---|---|---|---|---|---|---|
| PP_N | 5 | 0.7 | 0.0 | 0.0 | 0.0 | 0.0 | 0.0 | 0.0 | 0.0 |
| PP_N | 6 | 0.0 | 0.0 | 5.1 | 0.0 | 0.6 | 0.0 | 0.3 | 0.2 |
| PP_N | 7 | 0.0 | 0.0 | 0.0 | 0.9 | 0.8 | 0.0 | 0.5 | 0.5 |
| PP_N | 8 | 0.0 | 2.2 | 4.2 | 0.0 | 0.9 | 2.8 | 3.3 | 1.1 |
| PP_N | 9 | 0.0 | 2.0 | 3.8 | 45.9 | 2.2 | 3.9 | 5.1 | 3.7 |
| PP_N | 10 | 0.0 | 1.6 | 0.0 | 1.2 | 0.6 | 1.8 | 1.4 | 0.3 |
| PP_N | 11 | 0.5 | 0.0 | 0.8 | 0.5 | 0.0 | 0.3 | 0.3 | 0.0 |
| PP_N | 12 | 0.0 | 0.0 | 0.0 | 0.0 | 0.0 | 0.0 | 0.0 | 0.0 |
| PP_MD | 8 | 0.0 | 0.0 | 0.0 | 0.2 | 0.0 | 0.0 | 0.0 | 0.0 |
| PP_MD | 9 | 0.0 | 0.0 | 0.0 | 0.0 | 0.0 | 0.0 | 0.0 | 0.8 |
| PP_MD | 10 | 0.8 | 0.0 | 0.3 | 0.1 | 0.2 | 0.0 | 0.0 | 1.3 |
| PP_MD | 11 | 2.7 | 0.0 | 6.0 | 2.2 | 0.5 | 0.6 | 0.6 | 1.4 |
| PP_MD | 12 | 0.5 | 0.0 | 1.1 | 5.8 | 0.3 | 1.3 | 0.3 | 1.2 |
| PP_MD | 13 | 0.6 | 0.0 | 1.1 | 2.5 | 0.5 | 0.5 | 1.1 | 0.8 |
| PP_MD | 14 | 0.8 | 0.0 | 1.0 | 20.0 | 0.6 | 0.0 | 0.9 | 1.0 |
| PP_MD | 15 | 0.7 | 0.0 | 0.8 | 3.1 | 0.0 | 0.0 | 0.7 | 0.1 |
| PP_MD | 16 | 1.8 | 0.0 | 0.6 | 8.3 | 0.0 | 0.0 | 0.0 | 0.0 |
| PP_MD | 17 | 0.3 | 0.4 | 0.9 | 2.7 | 0.2 | 0.0 | 0.0 | 0.0 |
| PP_MD | 18 | 0.9 | 0.3 | 0.9 | 7.4 | 0.0 | 0.0 | 0.0 | 0.0 |
| PP_MD | 19 | 0.2 | 0.0 | 0.2 | 4.5 | 0.6 | 0.2 | 0.1 | 0.0 |
| PP_MD | 20 | 0.3 | 0.0 | 0.4 | 2.5 | 0.2 | 0.0 | 0.0 | 0.0 |
| PP_MD | 21 | 0.0 | 0.0 | 0.0 | 1.1 | 0.0 | 0.0 | 0.0 | 0.0 |
| PP_MD | 22 | 0.0 | 0.0 | 0.0 | 0.0 | 0.0 | 0.0 | 0.0 | 0.0 |

The key features of the respective samples are summarized in the following Table, in which n_tot means total n-paraffins and n-olefins and i_tot means total i-parffins and i-olefins:

TABLE 3

| Component | PE_N | PE_MD | PP_N | PP_MD |
|---|---|---|---|---|
| nP | 20.9 | 27.7 | 1.2 | 9.6 |
| iP | 0.6 | 0.0 | 5.8 | 0.7 |
| nP/iP | 34.8 | — | 0.2 | 13.7 |
| nO | 33.5 | 32.8 | 13.9 | 13.3 |
| iO | 6.6 | 12.4 | 48.5 | 60.4 |
| iO/nO | 0.2 | 0.4 | 3.5 | 4.5 |
| n_tot | 54.4 | 60.5 | 15.1 | 22.9 |
| i_tot | 7.2 | 12.4 | 54.3 | 61.1 |
| n_tot/i_tot | 7.6 | 4.9 | 0.3 | 0.4 |
| nO | 33.5 | 32.8 | 13.9 | 13.3 |
| iO | 6.6 | 12.4 | 48.5 | 60.4 |
| nO + iO | 40.1 | 45.2 | 62.4 | 73.7 |
| diO | 1.6 | 5.0 | 5.1 | 3.1 |
| nO + iO + diO | 41.7 | 50.2 | 67.5 | 76.8 |
| (nO + iO)/diO | 25.1 | 9.0 | 12.2 | 23.8 |
| oxygen content | 0.83 | 0.35 | 1.57 | 1.62 |

Further, a fossil naphtha (FN) co-feed having the following PIONA analysis results was provided.

TABLE 4

| Sample | c. no | nP | iP | nO | iO | diO | N | A |
|---|---|---|---|---|---|---|---|---|
| FN | 4 | 0.16 | 0.00 | 0.00 | 0.00 | 0.00 | 0.00 | 0.00 |
| FN | 5 | 13.71 | 10.83 | 0.00 | 0.00 | 0.00 | 2.03 | 0.00 |
| FN | 6 | 26.08 | 24.91 | 0.00 | 0.00 | 0.00 | 18.56 | 2.02 |
| FN | 7 | 0.00 | 1.34 | 0.00 | 0.00 | 0.00 | 0.00 | 0.36 |

### Examples 1 and 2 as well as Comparative Examples 1 and 2

The LWP-based feeds PE_N, PE_MD, PP_N and PP_MD were blended with the fossil naphtha co-feed to give feedstocks (total cracker feeds) each comprising 20 wt.-% waste-plastic-derived feed and 80 wt.-% fossil naphtha feed.

Steam cracking experiments illustrating certain embodiments of the present invention were carried out on a bench scale equipment. The main parts of the steam cracking unit, the analytical equipment and the calibration procedure used in these Examples and Comparative Examples have been described in detail in the following publications K. M. Van Geem, S. P. Pyl, M. F. Reyniers, J. Vercammen, J. Beens, G. B. Marin, On-line analysis of complex hydrocarbon mixtures using comprehensive two-dimensional gas chromatography, Journal of Chromatography A. 1217 (2010) 6623-6633 and J. B. Beens, U. A. T. Comprehensive two-dimensional gas chromatography—a powerful and versatile technique. Analyst. 130 (2005) 123-127.

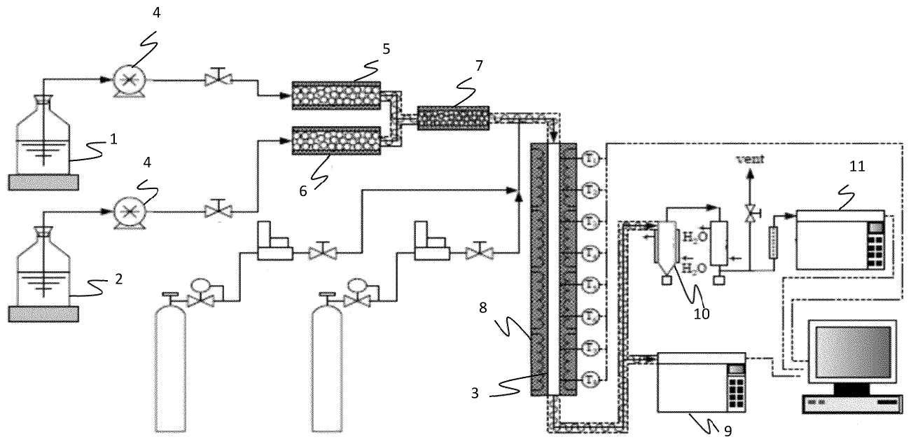

The bench scale equipment is described with reference to FIG. 1. The feed section controls the supply of the steam cracking feedstock and the water from reservoirs 1 and 2, respectively, to the reactor coil 3. The flow of liquids was regulated by coriolis flow meter controlled pumps 4 (Bronkhorst, The Netherlands) equipped with Bronkhorst™ CORI-FLOW™ series mass flow metering instruments to provide high accuracy: ±0.2% of reading. CORI-FLOW™ mass flow metering instruments utilizes an advanced Coriolis type mass flow sensor to achieve reliable performance, even with changing operating conditions, e.g. pressure, temperature, density, conductivity and viscosity. The pumping frequency was automatically adjusted by the controller of the CORI-FLOW™ flow metering instrument. The mass flow rate, which contrary to the volume flow rate is not affected by changes in temperature or pressure, of all streams was measured every second, i.e. substantially continuously. Steam was used as a diluent and was heated to the same temperature as the evaporated feedstock. Both the feedstock and the steam were heated in electrically heated ovens 5 and 6, respectively. Downstream from ovens 5 and 6, the feedstock and the steam were mixed in an electrically heated oven 7 filled with quartz beads, which enabled an efficient and uniform mixing of feedstock and the diluent prior to entering the reactor coil 3. The mixture of feedstock and diluent steam entered the reactor coil 3 placed vertically in a rectangular electrically heated furnace 8. Eight thermocouples T positioned along the axial reactor coordinate measured the process gas temperature at different positions. The rectangular furnace 8 was divided into eight separate sections which could be controlled independently to set a specific temperature profile. The pressure in the reactor coil 3 was controlled by a back pressure regulator (not shown) positioned downstream from the outlet of the reactor coil 3. Two pressure transducers (not shown), placed at the inlet and outlet of the reactor, indicated the coil inlet (CIP) and the coil outlet pressure (COP), respectively. At the reactor outlet, nitrogen was injected to the reactor effluent as an internal standard for analytical measurements and to a certain extent contribute to the quenching of the reactor effluent. The reactor effluent was sampled online, i.e. during operation of the steam cracking setup, at a high temperature (350° C.). Namely, via a valve-based sampling system and uniformly heated transfer lines a gaseous sample of the reactor effluent was injected into a comprehensive two-dimensional gas chromatograph (GC×GC) 9 coupled to a Flame Ionization detector (FID) and a Mass Spectrometer (MS). A high temperature 6-port 2-way sampling valve of the valve-based sampling system was placed in an oven, where the temperature was kept above the dew point of the effluent sample. Further downstream the reactor effluent was cooled to approximately 80° C. Water and condensed heavier products (pyrolysis gasoline (PyGas) and pyrolysis fuel oil (PFO)) were removed by means of a knock-out vessel and a cyclone 10, while the remainder of the effluent stream was sent directly to a vent. Before reaching the vent, a fraction of the effluent was withdrawn for analysis on a Refinery Gas Analyser (RGA) 11. After removal of all remaining water using a water-cooled heat exchanger and dehydrator, this effluent fraction was injected automatically onto the so-called Refinery Gas Analyser (RGA) 11 using a built-in gas sampling valve system (80° C.).

The steam cracking experiments were conducted at a dilution (feed rate of H2O diluent [kg/h]/feed rate of feed and co-feed [kg/h]) of 0.5 (also referred to as H₂O:HC ratio), a COP of 1.7 bar (absolute) and the COT being varied as shown in the table below. The steam cracking effluent was analysed and the product yields of the main products as well as main side products and waste products are as follows (values in wt.-%):

TABLE 5

| LWP-based | | Ex. 1 | CEx. 1 | Ex. 2 | CEx. 2 |
|---|---|---|---|---|---|
| feed | COT [° C.] | PE_N | PP_N | PE_MD | PP_MD |
| C₂H₄ | 820 | 22.3 | 18.5 | 22.2 | 18.3 |
| C₂H₄ | 850 | 26.7 | 23.9 | 27.5 | 24.2 |
| C₂H₄ | 880 | 30.0 | 26.4 | 30.7 | 27.8 |
| C₃H₆ | 820 | 15.1 | 15.4 | 14.9 | 15.6 |
| C₃H₆ | 850 | 15.7 | 15.7 | 16.3 | 16.3 |
| C₃H₆ | 880 | 13.2 | 15.0 | 13.6 | 13.9 |
| i-C₄H₈ | 820 | 2.2 | 4.5 | 2.5 | 4.1 |
| i-C₄H₈ | 850 | 2.3 | 3.6 | 2.3 | 3.5 |
| i-C₄H₈ | 880 | 1.6 | 3.9 | 1.6 | 2.4 |
| C₂H₂ | 820.0 | 0.23 | 0.20 | 0.21 | 0.20 |
| C₂H₂ | 850.0 | 0.43 | 0.41 | 0.47 | 0.42 |
| C₂H₂ | 880.0 | 0.78 | 0.61 | 0.77 | 0.71 |
| PD | 820.0 | 0.24 | 0.36 | 0.24 | 0.33 |
| PD | 850.0 | n.d. | 0.53 | 0.41 | 0.52 |
| PD | 880.0 | 0.47 | 0.54 | 0.47 | 0.56 |
| MeAc | 820.0 | 2.14 | 2.76 | 2.46 | 2.66 |
| MeAc | 850.0 | 1.19 | 1.22 | 1.24 | 1.23 |
| MeAc | 880.0 | 0.55 | 2.62 | 0.59 | 0.67 |
| CO₂ | 820 | 0.08 | 0.01 | 0.02 | 0.02 |
| CO₂ | 850 | 0.01 | 0.03 | 0.03 | 0.03 |
| CO₂ | 880 | 0.05 | 0.03 | 0.02 | 0.03 |
| CO | 820 | 0.28 | 0.37 | 0.09 | 0.42 |
| CO | 850 | 0.25 | 0.63 | 0.14 | 0.74 |
| CO | 880 | 0.36 | 0.59 | 0.18 | 0.69 |

As can be seen from the above data, total cracker feeds comprising LWP-based feeds (PE_N and PE_MD) having a low (or even very low) ratio of i-olefins to n-olefins tend to increased ethylene yields while total cracker feeds comprising LWP-based feeds having an i-olefin to n-olefin ratio exceeding 3.0 (PP_N) or even being as high as 4.6 (PP_D) tend to decreased ethylene yield while showing increased isobutene yields. Additionally, propylene yields are generally favoured by the LWP-based feeds having high i-olefin content but this effect can be compensated by varying COT so that the LWP feed and the total cracking feed of the present invention can be optimised towards high ethylene and propylene yields. Since ethylene is a more valuable product than isobutene (i-C₄H₈), the LWP feed according to the present invention is clearly more favourable in terms of value of the resulting cracking products in this respect.

Similarly, undesired products, namely acetylene (C₂H₂), propadiene (PD) and methylacetylene (MeAc; propyne) are less abundant when employing the feed of the present invention. Specifically, it can be seen that feeds having a high content of i-olefins, namely PP_N and PP_MD, are particularly prone to form MeAc which follows the propylene product stream. This product, if untreated, causes fouling, oligomerisation/polymerisation of the product stream and may also act as a polymerisation catalyst poison.

The results also show that formation of CO (carbon monoxide) is increased in i-olefin rich feeds while the formation of CO₂ is hardly influenced by the feed. CO is a severe poison for polymerisation catalysts and requires removal from ethylene and propylene streams. The Comparative Examples exhibit worse performance in that they have substantially higher CO formation than the Examples of the invention.

Moreover, it can be seen from the following table that coking tendency is much lower for low i-olefin content naphtha range fraction, while it is even increased for naphtha range fraction based on i-olefin rich feed, as determined in a separate coking experiment where the amount of coke which was deposited during a straight cracking duration of 6 hours at 650° C. has been quantified. The quantification was performed using an infrared meter which measures the volumetric concentrations of CO and CO₂ that were produced while decoking the reactor coil using the following decoking procedure:
Temperature in all heating zones: 850° C.
Flow of steam: 0.05 kg/h
Flow of air: 0.05 kg/h
After the CO₂ value drops below 1 vol. %: water flow is stopped and the reactor temperature is increased to 900° C. in all zones in order to thoroughly decoke the reactor.
Decoking was finished once the vol % of CO and CO2 in the effluent went to zero.

TABLE 6
Coking Rate

| Component | PE_N | PP_N | PE_MD | PP_MD |
|---|---|---|---|---|
| Coke (g/6 h) | 0.04 | 0.11 | 0.08 | 0.05 |

### Reference Examples 1 to 5

Model feeds comprising mainly n-decane (nC10) were produced according to the following table to illustrate the effects of embodiments of the invention. These model feeds, while not being based on liquefied waste plastic, show the influence of specific types of components, namely mono-olefins, non-conjugated di-olefins, and conjugated di-olefins.

TABLE 7

| RefEx | Paraffin | Monoolefin | Diolefin | Description |
|---|---|---|---|---|
| 1 | nC10 | None | None | nP |
| 2 | nC10 | 40% 1-decene | None | nO |
| 3 | nC10 | 30% 1-decene | 10% 1,7-octadiene | diO[NC] |
| 4 | nC10 | 30% 1-decene | 10% 2,5-dimethyl-2,4- | diO[C] |
| | | | hexadiene | |
| 5 | nC10 | 40% 2-methyl- | None | iO |
| | | 2-butene | | |

These model feeds were then cracked at 750° C., 800° C. and 880° C., with H₂O:HC ratio of 0.5 at a coil outlet pressure of 1.7 bar.

The steam cracking effluent was analysed and the yields of products as well as side products are shown in the table below. In addition, after cracking at the three temperatures (from low to high temperature) the reactor was then decoked and the amount recorded in g.

TABLE 8

| COT | Component yield | | | | | |
|---|---|---|---|---|---|---|
| [° C.] | [wt.-%] | nP | nO | iO | diO[NC] | diO[C] |
| 750 | C2H4 | 20.52 | 22.68 | 13.28 | 20.01 | 21.33 |
| 800 | C2H4 | 33.14 | 32.75 | 21.78 | 31.18 | 31.18 |
| 880 | C2H4 | 43.1 | 40.24 | 30.34 | 38.5 | 37.12 |
| 750 | C3H6 | 8.06 | 12.61 | 7.7 | 13.98 | 14.19 |
| 800 | C3H6 | 16.61 | 14.58 | 11.78 | 10.83 | 17.96 |
| 880 | C3H6 | 13.81 | 10.96 | 10.47 | 10.86 | 12.79 |
| 750 | i-C4H8 | 0.2 | 0.09 | 4.3 | 0.64 | 0.97 |
| 800 | i-C4H8 | 1.35 | 0.1 | 5.34 | 0.31 | 1.31 |
| 880 | i-C4H8 | 1.03 | 0.09 | 2.67 | 0.41 | 0.97 |
| 750 | C2H2 | 0.15 | 0.08 | 0.02 | 0.05 | 0.05 |
| 800 | C2H2 | 0.16 | 0.23 | 0.13 | 0.29 | 0.18 |
| 880 | C2H2 | 0.76 | 0.84 | 0.79 | 0.8 | 0.75 |
| 750 | MeAc | 0 | 0.09 | 0.12 | 0.04 | 0.06 |
| 800 | MeAc | 0.02 | 0.15 | 0.14 | 0.19 | 0.01 |
| 880 | MeAc | 0.04 | 0.33 | 0.3 | 0.31 | 0.33 |
| 750 | PD | 0.05 | 0.08 | 0.11 | 0.05 | 0.06 |
| 800 | PD | 0.11 | 0.12 | 0.32 | 0.12 | 0.13 |
| 880 | PD | 0.28 | 0.2 | 0.66 | 0.19 | 0.27 |
| 750 | C2H2\|MeAc\|PD | 0.21 | 0.24 | 0.25 | 0.14 | 0.17 |
| 800 | C2H2\|MeAc\|PD | 0.28 | 0.5 | 0.59 | 0.59 | 0.32 |
| 880 | C2H2\|MeAc\|PD | 1.08 | 1.37 | 1.75 | 1.29 | 1.35 |
| — | Coke (g) | 9.8 | 8.8 | 11.9 | 12.8 | 17.1 |

The nO model feed is a model for a degassed naphtha range LWP feed having a total olefins content of 40 wt.-%, whereas the iO model feed is a model for a degassed naphtha range LWP feed having a total olefins content of 40 wt.-% and having a high (infinite) i-olefins/n-olefins ratio. It can be clearly seen that a high content of i-olefins results in significant reduction of ethylene yield and, in addition causes more coking as well as significantly higher yield of propadiene. Comparison between n-diO (non-conjugated) and i-diO (conjugated) shows that the conjugated di-olefin-containing model feed clearly has the highest coking tendency.

1.-18. (canceled)

19. A method for upgrading liquefied waste plastic, wherein the method comprises:
(a) providing a liquefied waste plastic feed;
(b) thermally cracking the liquefied waste plastic feed in a thermal cracking reactor; and
(c) separating at least a fraction including ethylene from an effluent of the thermal cracking reactor;
wherein
the liquefied waste plastic feed has a content ratio of i-olefins to n-olefins of 3.0 or less.

20. The method according to claim 19, wherein the liquefied waste plastic feed has a content ratio of i-olefins to n-olefins of at least one or more of 2.5 or less, 2.0 or less, 1.5 or less, 1.0 or less, 0.80 or less, 0.60 or less, and/or 0.50 or less.

21. The method according to claim 19, wherein the liquefied waste plastic feed has a total content of i-olefins, n-olefins, i-paraffins and n-paraffins of at least one or more of at least 40 wt.-%, at least 50 wt.-%, at least 55 wt.-%, at least 60 wt.-%, at least 65 wt.-% and/or at least 70 wt.-%.

22. The method according to claim 19, wherein the liquefied waste plastic feed has a total content of gaseous (NTP) components of at least one or more of 1.5 wt.-% or less, 1.0 wt.-% or less, and/or 0.5 wt.-% or less.

23. The method according to claim 19, wherein the liquefied waste plastic feed has a total olefins content of at least one or more of 10 wt.-% or more, 15 wt.-% or more, 20 wt.-% or more and/or 30 wt.-% or more; and has a total olefins content of at least one or more of 90 wt.-% or less, 80 wt.-% or less, 70 wt.-% or less and/or 60 wt.-% or less.

24. The method according to claim 19, wherein the liquefied waste plastic feed has a content ratio of monoolefins to diolefins of at least one or more of 10 or more, 12 or more, 15 or more and/or 20, or more.

25. The method according to claim 19, wherein the liquefied waste plastic feed has a content ratio of i-paraffins to n-paraffins of at least one or more of 2.0 or less, 1.7 or less, 1.5 or less, 1.0 or less, 0.90 or less, 0.80 or less, 0.70 or less, 0.60 or less, 0.50 or less, 0.40 or less, 0.30 or less, 0.20 or less, 0.15 or less, 0.10 or less, 0.09 or less, 0.08 or less, 0.07 or less, 0.06 or less, 0.05 or less, and/or 0.04 or less.

26. The method according to claim 19, wherein the liquefied waste plastic feed has a total paraffins content of at least one or more of 5 wt.-% or more, 10 wt.-% or more, 12 wt.-% or more, 15 wt.-% or more, 17 wt.-% or more, 19 wt.-% or more, and/or 20 wt.-% or more.

27. The method according to claim 19, wherein the liquefied waste plastic feed has a total content of n-paraffins and n-olefins of at least one or more of 20 wt.-% or more, 23 wt.-% or more, 25 wt.-% or more, 27 wt.-% or more, and/or 30 wt.-% or more; and/or
wherein the liquefied waste plastic feed has a content ratio of total n-paraffins and n-olefins to total i-paraffins and i-olefins ([nP]+[nO])/([iP]+[iO]) of at least one or more of 0.40 or more, 0.50 or more, 0.60 or more, 0.70 or more, 0.80 or more, 0.90 or more, 1.00 or more, 1.20 or more, 1.40 or more, 1.60 or more, 1.80 or more, 2.00 or more, 2.50 or more, and/or 3.00 or more; and/or
wherein the liquefied waste plastic feed has a total content of n-olefins, i-olefins and diolefins ([nO]+[iO]+[diO]) of at least one or more of 60 wt.-% or less, 55 wt.-% or less, and/or 50 wt.-% or less; and/or
wherein the liquefied waste plastic feed has a content ratio of total n-olefins and i-olefins to diolefins (([nO]+[iO])/[diO]) of at least one or more of 5 or more, 8 or more, and/or 10 or more; and/or
wherein the liquefied waste plastic feed has a total content of i-paraffins and i-olefins of at least one or more of 50 wt.-% or less, 40 wt.-% or less, 30 wt.-% or less, and/or 20 wt.-% or less.

28. The method according to claim 19, wherein the liquefied waste plastic feed has a total content of C9 i-olefins of at least one or more of 10 wt.-% or less, 8 wt.-% or less, 6 wt.-% or less and/or 5 wt.-% or less; and/or
wherein the liquefied waste plastic feed has a content of diolefins of at least one or more of 5.0 wt.-% or less, 4.0 wt.-% or less, 3.0 wt.-% or less, 2.5 wt.-% or less, 2.0 wt.-% or less, 1.5 wt.-% or less, and/or 1.0 wt.-% or less; and/or
wherein the liquefied waste plastic feed has a content of conjugated diolefins of at least one or more of 5.0 wt.-% or less, 4.0 wt.-% or less, 3.0 wt.-% or less, 2.0 wt.-% or less, 1.0 wt.-% or less, 0.75 wt.-% or less, and/or 0.50 wt.-% or less.

29. The method according to claim 19, wherein the liquefied waste plastic feed has a content of n-olefins of at least one or more of 2 wt.-% or more, 5 wt.-% or more, 10 wt.-% or more, 15 wt.-% or more, 20 wt.-% or more, and/or 25 wt.-% or more.

30. The method according to claim 19, wherein thermal cracking in step (b) comprises:
steam cracking.

31. The method according to claim 19, comprising:
carrying out the thermal cracking in step (b) in a presence of at least one or more of a co-feed, a hydrocarbon co-feed, a fossil naphtha co-feed, and/or a fossil naphtha feed selected from full range naphtha and light naphtha.

32. The method according to claim 19, wherein the step (a) of providing the liquefied waste plastic feed comprises:
at least one pre-treatment stage in which liquefied waste plastic or a fraction thereof is subjected to pre-treatment, and wherein the pre-treatment includes subjecting the liquefied waste plastic or a fraction thereof to at least one treatment selected from the group consisting of drying, desalting, reactive extraction, solvent extraction, adsorption, oxidation, reduction, separation of solid particles from the liquefied waste plastic, separation of gaseous (NTP) components from the liquefied waste plastic, and selective and/or mild hydrogenation of the liquefied waste plastic.

33. The method according to claim 19, wherein the liquefied waste plastic feed has a total content of i-olefins, n-olefins, i-paraffins and n-paraffins in a range of at least one or more of 40 wt.-% to 100 wt.-%, 50 wt.-% to 100 wt.-%, 55 wt.-% to 100 wt.-%, 60 wt.-% to 100 wt.-%, 65 wt.-% to 100 wt.-%, and/or 70 wt.-% to 100 wt.-%.

34. A liquefied waste plastic feed for thermal cracking, wherein the liquefied waste plastic feed has a content ratio of i-olefins to n-olefins of 3.0 or less.

35. The liquefied waste plastic feed according to claim 34, wherein the liquefied waste plastic feed has a content ratio of i-olefins to n-olefins of at least one or more of 2.5 or less, 2.0 or less, 1.5 or less, 1.0 or less, 0.80 or less, 0.60 or less, and/or 0.50 or less.

38. A liquefied waste plastic feed for thermal cracking, wherein the liquefied waste plastic feed is a pre-treated liquefied waste plastic obtained from a process comprising:
depolymerisation of polyethylene-rich waste plastic, or a fraction thereof.
